(19) United States
(12) Patent Application Publication
(10) Pub. No.: US 2021/0261902 A1
(43) Pub. Date: Aug. 26, 2021

(54) BIOLOGICAL SAMPLE WARMING METHOD, BIOLOGICAL SAMPLE WARMING VESSEL, AND KIT FOR WARMING BIOLOGICAL SAMPLE

(71) Applicant: TWO CELLS Co., Ltd., Hiroshima (JP)

(72) Inventors: Shunsuke TANIGAWA, Hiroshima (JP); Masanori NAKASA, Hiroshima (JP); Jin Chang SHAO, Hiroshima (JP); Yukio KATO, Hiroshima (JP)

(21) Appl. No.: 17/254,108

(22) PCT Filed: Jun. 28, 2019
(86) PCT No.: PCT/JP2019/025962

§ 371 Date: Dec. 18, 2020

(30) Foreign Application Priority Data
Jun. 28, 2018 (JP) 2018-123502

(51) Int. Cl. C12M 1/02; A01N 1/02

(52) U.S. Cl. CPC C12M 41/18; A01N 1/0284

(57) ABSTRACT

Provided is a simple, safe warming method which causes no or little damage to a biological sample. A method of warming a biological sample includes the steps of: i) putting a biological sample container (5) into a heat medium container (2), the biological sample container (5) having a biological sample disposed therein, the heat medium container (2) having a heat medium (4) disposed therein; and thereafter ii) closing an inlet (3) of the heat medium container (2), the inlet being an inlet through which the heat medium (4) is injected into the heat medium container (2); and iii) moving the heat medium (4) by moving the heat medium container (2) with the inlet (3) closed.

### TECHNICAL FIELD

The present invention relates to a method of warming a biological sample, a warming container for warming a biological sample, and a kit for warming a biological sample.

### BACKGROUND ART

Patent Literature 1 discloses an apparatus for thawing frozen cells. The apparatus is configured to thaw cryopreserved cells or tissue by heating the cryopreserved cells or tissue with a heater that heats the cryopreserved cells or tissue to a temperature above their melting point.

### CITATION LIST

### Patent Literature

[Patent Literature 1]
Published Japanese Translation of PCT International Application, Tokuhyo, No. 2017-526375 (Sep. 14, 2017)

### SUMMARY OF INVENTION

### Technical Problem

In the production of cell products for regenerative medicine and cellular research, a cell freezing technique is essential. Stably freezing and thawing cells without causing changes to the properties of cells make it possible to improve the productivity of cell products for regenerative medicine. It is also possible, in the cellular research, to acquire highly reliable data with few variations.

Various researches have been done on a cell freezing method, and it is possible for a user to select the most appropriate preservation solution depending on the type of cell. However, with regard to a method of thawing frozen cells, although users employ their own protocols, there is no established best method in terms of simplicity and safety.

One of the typically known conventional thawing methods is a method involving thawing frozen cells with use of a water bath. When raising the temperature of the frozen cells with use of a water bath, by bringing the frozen cells into contact with water which has been heated to about body temperature, it is possible to prevent the properties of the cells from changing because the cells are not subjected to high temperature. However, the water bath requires a large amount of water, and, in addition, the instrument occupies a large volume and is heavy. In addition, since a temperature about body temperature is employed, germs are likely to thrive in the water serving as a heat medium. In particular, in a case where, like cell products for regenerative medicine, frozen cells or processed cells are required to be thawed in an operating room or near the bedside where the cells are to be used, it is difficult to place a large instrument such as a water bath in the site, and contamination with a large amount of water is also an issue.

On the other hand, a conventional frozen cell thawing apparatus which employs a heater as a heat source and which thaws the cells via a solid heat medium, i.e., a heat block incubator (heat block), does not require a large amount of water and the apparatus is small in size. However, in a case where the temperature is set to 37° C. (which is substantially the same as the water bath), the heat block is lower in heat transfer efficiency than the water bath and requires more time to thaw the frozen cells, which results in an increased risk that the cells will be damaged due to, for example, concentration gradient and temperature gradient during thawing.

Furthermore, with regard to the apparatus for thawing frozen cells disclosed in Patent Literature 1, there can be a risk that the cells will be prone to damage because of the temperature of the heater higher than 37° C.

An aspect of the present invention was made in view of the above issues, and an object thereof is to provide a simple, safe warming method which causes no or little damage to a biological sample.

### Solution to Problem

In order to attain the above object, a warming method in accordance with an aspect of the present invention is a method of warming a biological sample, including the steps of: i) putting a biological sample container into a heat medium container, the biological sample container having a biological sample disposed therein, the heat medium container having a heat medium disposed therein; ii) closing an inlet of the heat medium container to prevent the heat medium from leaking out of the heat medium container, the inlet being an inlet through which the heat medium is injected into the heat medium container; and iii) moving the heat medium in the heat medium container with the inlet closed.

A warming container in accordance with an aspect of the present invention is a warming container for warming a biological sample, including: a heat medium container configured to have a heat medium disposed therein and have a biological sample container disposed therein, the biological sample container being configured to have a biological sample disposed therein; and a positioning part which is attached to the heat medium container and which is configured to keep the biological sample container in position.

A kit for warming a biological sample in accordance with an aspect of the present invention includes a heat medium container configured to have a heat medium and a biological sample container disposed therein, wherein: the heat medium container includes a positioning part configured to keep the biological sample container in position; and the biological sample container is configured to have a biological sample disposed therein.

### Advantageous Effects of Invention

An aspect of the present invention makes it possible to warm a biological sample in a simple, safe manner with no or little damage to the biological sample.

### BRIEF DESCRIPTION OF DRAWINGS

FIG. 1 schematically illustrates a warming container for use in a warming method in accordance with an aspect of the present invention.

FIG. 2 schematically illustrates a warming container for use in a warming method in accordance with another aspect of the present invention.

FIG. 3 schematically illustrates a warming container in accordance with a further aspect.

FIG. 4 schematically illustrates a warming container in accordance with a further aspect.

FIG. 5 schematically illustrates a warming container in accordance with a further aspect.

FIG. 6 schematically illustrates a warming container in accordance with a further aspect.

FIG. 7 schematically illustrates a warming container in accordance with a further aspect.

FIG. 8 schematically illustrates a kit for warming in accordance with an aspect of the present invention.

FIG. 9 shows an example of mixing by inversion.

FIG. 10 shows examples of a heat medium container and a biological sample container.

FIG. 11 shows an example of the dimensions of a resin film.

FIG. 12 shows an example of how a warming container is held by human hand.

FIG. 13 is an enlarged view of a part of FIG. 12.

FIG. 14 is a top view of an example of a warming container.

FIG. 15 shows an example of how a biological sample container is put in a heat medium container.

FIG. 16 is a chart showing the results of thawing.

FIG. 17 is a chart showing the results of thawing.

FIG. 18 is a chart showing the results of thawing.

FIG. 19 is a chart showing the proliferating ability of thawed cells.

FIG. 20 is a chart showing the proliferating ability of thawed cells.

FIG. 21 is a chart showing the proliferating ability of thawed cells.

FIG. 22 is a chart showing the results of thawing.

FIG. 23 is a chart showing the proliferating ability of thawed cells.

FIG. 24 shows charts showing the results of thawing.

FIG. 25 shows charts showing the proliferating ability of thawed cells.

### DESCRIPTION OF EMBODIMENTS

[Warming Method]

A warming method in accordance with an aspect of the present invention is a method of warming a biological sample, including the steps of: i) putting a biological sample container into a heat medium container, the biological sample container having a biological sample disposed therein, the heat medium container having a heat medium disposed therein; ii) closing an inlet of the heat medium container to prevent the heat medium from leaking out of the heat medium container, the inlet being an inlet through which the heat medium is injected into the heat medium container; and iii) moving the heat medium in the heat medium container with the inlet closed.

A warming method in accordance with a preferred aspect of the present invention is a method of warming a biological sample, including the steps of: i) putting a biological sample container into a heat medium container, the biological sample container having a biological sample disposed therein, the heat medium container having a heat medium disposed therein; and thereafter ii) closing an inlet of the heat medium container to prevent the heat medium from leaking out of the heat medium container, the inlet being an inlet through which the heat medium is injected into the heat medium container; and iii) moving the heat medium by moving the heat medium container with the inlet closed.

In the present specification, the term “biological sample” refers to a sample derived from a living body, and is preferably at least one selected from the group consisting of cells, cell masses, tissue, and tissue fragments.

Examples of cells as a biological sample include various types of useful cells. Examples of the cells include: mesenchymal stem cells (MSCs) derived from various types of tissue; iPS cells and cell lines derived therefrom; ES cells and cell lines derived therefrom; other stem cells such as hematopoietic stem cells and neural stem cells; cancer cells; vascular precursor cells; vascular cells; myoblasts; cells derived from umbilical cord; cartilage cells; osteoblastic cells; intervertebral disc cells; and genetically-modified cells.

Examples of tissue as a biological sample include various types of useful tissue. Examples of the tissue include bone marrow aspirate, umbilical cord blood, umbilical cord tissue, various types of cell fractions derived from bone marrow, adipose tissue fragments, sperm, ova, heterologous cadaveric or autologous cadaveric cartilage tissue, and bone tissue. Examples of tissue as a biological sample further include tissue derived from ES cells, tissue derived from iPS cells, and tissue for grafting including various types of cells prepared by tissue engineering.

In an aspect of the present invention, a biological sample to be warmed may be a biological sample in a frozen state or a biological sample in a non-frozen state. That is, a warming method in accordance with an aspect of the present invention can be used to thaw a frozen sample and, for example, can be used to allow cells and tissue which have been preserved by a non-freezing, low-temperature preservation method to return to room temperature or body temperature.

In the present specification, the “biological sample in a frozen state” may be referred to as “frozen sample”. The term “frozen sample” means a sample in a cryopreserved state. The frozen sample is preferably a sample which has been preserved at extremely low temperature such as not lower than −250° C. and not higher than −60° C. for a certain period of time such as several hours to several years or longer. In a warming method in accordance with an aspect of the present invention, a method of freezing a biological sample which is to be warmed is not particularly limited, and may be a known freezing method. The biological sample may be one that has been frozen with its three-dimensional structure maintained by, for example, a method using a scaffold, or may be one that has been cryopreserved with use of a cryopreservation solution. The cryopreservation solution may contain, for example, a fatty acid, a phospholipid, a surfactant, and/or the like. One of these may be contained alone or in combination of two or more.

In an aspect of the present invention, the “biological sample in a non-frozen state” is, for example, a biological sample which has been preserved at extremely low temperature for a certain period of time. Examples of such a biological sample include: the foregoing cells and tissue preserved by a non-freezing, low-temperature preservation method; and heterologous cadaveric or autologous cadaveric cartilage tissue and various types of cells for grafting preserved by a non-freezing, low-temperature preservation method.

The non-freezing, low-temperature preservation method means a method of preservation under a low-temperature environment in which freezing does not take place, such as a cool (5° C. to 10° C.) or chilling (0° C. to 5° C.) environment. The method involves preserving tissue, cells, a cell mass, or the like preferably immersed in an isotonic solution containing, e.g., a culture medium, lactated Ringer's solution, physiological saline, and/or the like.

In the present specification, the term “warming” means applying heat to a biological sample to raise temperature. For example, in a case where the biological sample is a frozen sample, the term “warming” means thawing the biological sample with the heat applied. In the present specification, the term “thaw” means that the solid phase of a frozen sample at least partially turns into a liquid phase, preferably means that the part of the frozen sample which was a liquid phase before freezing completely thaws and returns to the liquid phase.

In the present specification, cryopreservation and non-freezing, low-temperature preservation may be collectively referred to as “low-temperature preservation” for short.

(Step of Putting)

The step of putting involves putting a biological sample container into a heat medium container, the biological sample container having a biological sample disposed therein, the heat medium container having a heat medium disposed therein. In the present specification, the phrase “put a container B into a container A” means: (1) putting the container B into an inner space of the container A; or (2) wrapping, with the container A, the container B which is in contact with the outside surface of the container A. The above instance (2) further means that the container A is made of a deformable material. The following description will discuss the step of putting based on the instance (1) as an example.

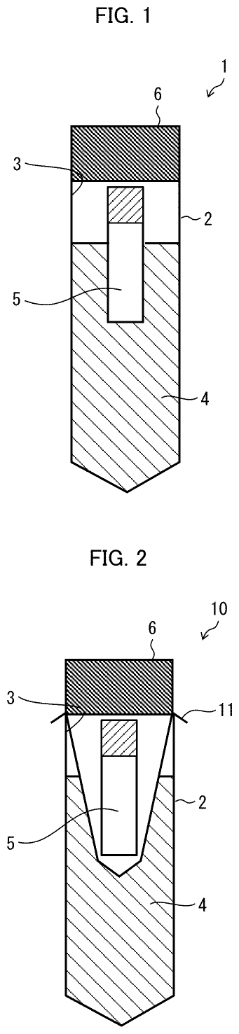

The heat medium container is configured to have a heat medium disposed therein, and has an opening serving as an inlet through which the heat medium is injected. An example of the heat medium container is discussed with reference to FIG. 1. FIG. 1 schematically illustrates a warming container for use in a warming method in accordance with an aspect of the present invention. A warming container 1 includes a heat medium container 2. The heat medium container 2 has an opening 3 for injection of a heat medium 4. The heat medium container 2 has disposed therein a biological sample container 5 which has a biological sample disposed therein. In the example illustrated in FIG. 1, the opening 3 is closed with a lid 6 (opening 3 is closed in the step of closing which will be described later).

The heat medium container need only be capable of having a heat medium disposed therein. As described later, the heat medium used in the present invention does not become hot; therefore, the heat medium container does not need to be heat resistant. Specifically, the heat medium container is more preferably a container made of a synthetic resin such as polyethylene, polypropylene, polystyrene, polyethylene terephthalate, or the like.

The heat medium container is preferably a container that is easily disposable after use, which makes it possible to prevent cross-contamination. Furthermore, the heat medium container is, for easy holding by human hand, preferably a tubular structure which has opposite ends one of which is a closed end and the other of which is an open end. It is more preferable that the tubular structure have, in a cross section perpendicular to the longitudinal direction of the tubular structure, a diameter of not less than 5 mm and not more than 200 mm. It is further preferable that the heat medium container can be carried by human hand. For example, a commercial centrifuge tube can be suitably used as the heat medium container.

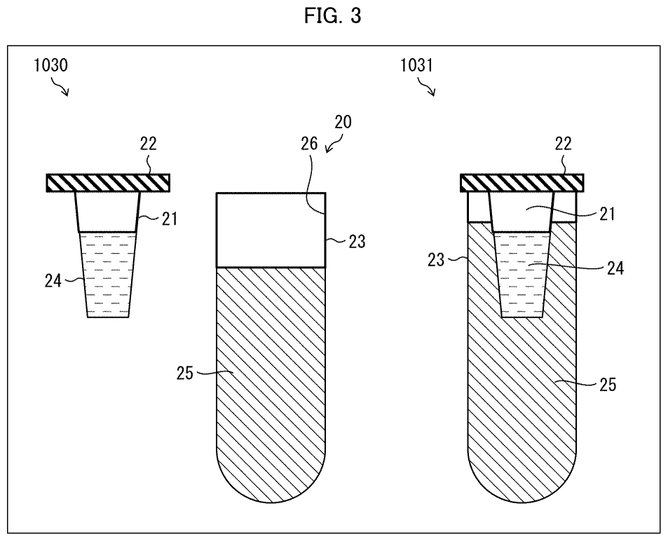

The heat medium container may further include, therein, a positioning part for keeping the biological sample container in position. An example of a heat medium container including a positioning part is discussed with reference to FIG. 2. FIG. 2 schematically illustrates a warming container 10 for use in a warming method in accordance with another aspect of the present invention. As illustrated in FIG. 2, the warming container 10 is different from the warming container 1 illustrated in FIG. 1 in that the warming container 10 includes a resin film 11 serving as a positioning part.

The resin film 11 is in the form of a pouch that can have the biological sample container 5 disposed therein. The resin film 11 is preferably an elastic film, examples of which include nitrile rubber, polyurethane, and natural rubber. The resin film 11 is provided such that the resin film 11 covers the opening 3, that the top of the pouch is folded over the edge of the opening 3, and that the resin film 11 is in close contact with the heat medium container 2. The position where the biological sample container 5 is disposed is isolated by the resin film 11 so that the heat medium 4 and the biological sample container 5 do not physically contact each other. Note that, in the example illustrated in FIG. 2, the opening 3 is closed with the lid 6 (opening 3 is closed in the step of closing).

Since the heat medium container includes the positioning part as described above, the movement of the biological sample container within the heat medium container is restricted. This makes it possible to eliminate the likelihood that the biological sample container will be displaced greatly in the step of moving (described later) and hit the lid or the like and that the container will be broken. Furthermore, in particular, since the positioning part has the function of preventing the physical contact between the heat medium 4 and the biological sample container 5 and therefore the heat medium and the biological sample container do not directly contact each other within the heat medium container, it is possible to reduce the risk that the heat medium will flow into the biological sample container and the biological sample will be contaminated.

The heat medium is put into the heat medium container through the opening. The heat medium need only be capable of exchanging heat with the biological sample in the biological sample container. The heat medium need only be a fluid having a heat capacity that can maintain temperature within a certain range for a certain period of time. Therefore, a fluid having higher specific heat capacity is more preferred, because high heat capacity is achieved even if the amount of the heat medium is small. In consideration of safety in addition to the above requirements, the heat medium is preferably at least one selected from the group consisting of water, isotonic solutions, and water which has an antibacterial agent dissolved therein. In a case where the biological sample is cells or a cell mass, the use of an isotonic solution as the heat medium makes it possible to reduce the risk that the cells will be damaged even if the heat medium and the biological sample contact each other.

The amount of the heat medium put into the heat medium container may be set such that the heat medium has a heat capacity that is sufficient to warm the biological sample, in consideration of the conditions such as the amount of the biological sample, the temperature of the heat medium, and/or the like.

(Variation of Step of Putting)

As described earlier, according to an aspect of the heat medium container, the biological sample container is disposed on the outside surface of the heat medium container (instance (2)). More specifically, in the instance (2), the heat medium container wraps the biological sample container. To this end, the heat medium container in the instance (2) is a container made of a flexible material.

(Order Estimation)

It is noted here that the amount of the heat medium and the capacity of the heat medium container can be estimated using order estimation based on to what degree the heat medium changes in temperature due to heat exchange between the biological sample and the heat medium, as described below. The type, amount, and temperature of the heat medium, the capacity of the heat medium container, and/or the like may be designed on the basis of the results of such order estimation.

For easy calculation, assume that the biological sample in the biological sample container and the heat medium have the same specific heat capacity, specific gravity, melting point, and the like as those of water or ice. The following description discusses an example in which the temperature change of the heat medium in the following case is calculated: the weight of the biological sample is 1 g, the weight of the heat medium is 40 g, the initial temperature of the biological sample is −80° C., and the initial temperature of the heat medium is 24° C. However, the present invention is not limited to such values. Note that the order estimation is based on the assumption that the heat medium does not exchange heat except with the biological sample, and the heat of the heat medium is entirely used by the biological sample to change in state and change in temperature. The temperature change and heat transfer in the system are estimated using order estimation using the following three-step calculation. In the following description, the “×” symbol means multiplication, the “/” symbol means division, and the “\hat{ }” symbol means power. Furthermore, in the following equations, a physical quantity Q expressed in unit “u” is expressed in the form of “Q [u]”.

<i: Process in which Biological Sample Changes from −80° C. Solid to 0° C. Solid>

Since the biological sample is in solid state, it is inferred that the specific heat capacity of the biological sample is near the specific heat capacity of ice (2.1 kJ K\hat{ }-1 kg\hat{ }-1). Therefore, in this process, the biological sample receives, in accordance with the following equation (1), the heat of the following equation (2) from the heat medium:

(Thermal energy)=(mass)×(specific heat capacity)×(temperature change) (Equation 1),

Q1 [kJ]=0.001×2.1×80=0.168 (Equation 2).

It is inferred that, as a result, heat of Q1 [kJ] is removed from the heat medium, and a temperature change occurs in accordance with the following equation (3):

(Temperature change)=(thermal energy)/((mass)×(specific heat capacity)) (Equation 3).

Since the heat medium is in liquid state, it is inferred that the specific heat capacity of the heat medium is near the specific heat capacity of water (4.2 kJ K\hat{ }-1 kg\hat{ }-1). Therefore, the following temperature change occurs in the heat medium:

ΔT1 [K]=(−0.168)/(0.04×4.2)=−1.0 (Equation 4).

Specifically, the temperature T of the heat medium, after going through the process i, is about 23° C. The initial temperature of the heat medium is 24° C. The heat medium does not reach its freezing point even if such a degree of temperature change occurs. Furthermore, this degree of temperature change is so small that it can be ignored during actual use, during which heat exchange with an external environment can also occur.

<ii: Process in which Biological Sample Changes from Solid to Liquid>

In the process in which the biological sample changes from solid to liquid, the biological sample needs to obtain, from the heat medium, thermal energy that corresponds to heat of fusion. The necessary thermal energy is calculated using the following equation (5):

(Thermal energy)=(mass)×(heat of fusion) (Equation 5).

It is inferred that the heat of fusion is near the heat of fusion of ice (335 kJ kg\hat{ }-1); therefore, in this process, the biological sample receives the following heat from the heat medium:

Q2 [kJ]=(0.001)×(335)=0.335 (Equation 6).

Therefore, the heat of Q2 is removed from the heat medium. As a result, similarly to the foregoing Equation 4, the following temperature change occurs in the heat medium:

ΔT2 [K]=(−0.335)/(0.04×4.2)=−1.99 (Equation 7).

Specifically, the temperature of the heat medium, after going through the processes i and ii, is about 21° C. This degree of temperature change is so small that it can be ignored during actual use, during which heat exchange with an external environment can also occur, because a change of heat is also very small.

<iii: Process in which Biological Sample Changes from 0° C. Liquid to Liquid Having Equilibrium Temperature>

According to the law of conservation of heat, an equilibrium temperature Too, resulting when a fluid having a temperature T, a mass M, and a specific heat capacity C and a fluid having a temperature T′, a mass M′, and a specific heat capacity C′ are brought into contact with each other, can be calculated using the following equation, assuming that no phase change occurs:

T∞=(M×C×T+M′×C′×T′)/(M×C+M′×C′) (Equation 8).

It is noted here that the biological sample and the heat medium can be regarded as having the same specific heat capacity. Therefore, when the absolute zero is expressed as T0 [° C.], 0° C.=−T0 [K] holds, and the above Equation 8 can be simplified as below.

$$\begin{matrix}{T}_{∞} [ K ] = ( M \times T [ K ] + {M}^{′} \times {T}^{′} [ K ] ) / ( M + {M}^{′} ) \\ = ( M \times ( T {[}^{°} C . ] + T 0 [ K ] ) + {M}^{′} \times \\ ( {T}^{′} {[}^{°} C . ] + T 0 [ K ] ) ) / ( M + {M}^{′} ) \\ = ( M \times T {[}^{°} C . ] + {M}^{′} \times {T}^{′} {[}^{°} C . ] ) / \\ ( M + {M}^{′} ) + T 0 [ K ]\end{matrix} \qquad ( Equation {8}^{′} )$$

Therefore, if the initial temperature of the biological sample in the process iii is 0° C., the following equation holds:

$$\begin{matrix}{T}_{∞} {[}^{°} C . ] = ( M \times T {[}^{°} C . ] + {M}^{′} \times {T}^{′} {[}^{°} C . ] ) / \\ ( M + {M}^{′} ) \\ = ( M [ g ] \times T {[}^{°} C . ] ) / ( M [ g ] + {M}^{′} [ g ] ) .\end{matrix} \qquad ( Equation {8}^{″} )$$

The equilibrium temperature in this process is therefore:

T∞[° C.]=(21×40+0×1)/(40+1)=20.5 (Equation 8′″).

Therefore, a temperature change ΔT3 that occurs in the heat medium in this process is:

ΔT3 [K]=20.5−21=−0.5 (Equation 9).

Furthermore, in this process, similarly to the foregoing Equation 1, the biological sample receives heat of the following equation from the heat medium:

Q3 [kJ]=0.001×4.2×20.5=0.086 (Equation 10).

This means that heat of Q3 [kJ] is removed from the heat medium. This degree of temperature change is so small that it can be ignored during actual use, during which heat exchange with an external environment can also occur, because a change of heat is also very small.

According to the above-described order estimation, the temperature T of the heat medium changes from 24° C. to 20.5° C. due to the fusion in the processes i to iii. Therefore, the temperature decreases by 3.5° C. in total. However, in practice, there is a heat transfer between the heat medium and an external environment which was ignored in the order estimation; therefore, the above-mentioned temperature decrease of the heat medium is so small that it can be ignored during actual use.

As such, the type, amount, and thermal conditions such as the temperature of the heat medium can be designed so that a temperature change will be substantially the same as the foregoing temperature change, on the basis of the conditions in which substantially the same warming time as in the case of a water bath was achieved. Specifically, the amount of the heat medium is not limited, provided that a forced flow occurs in the step of moving (described later), that the heat medium and the biological sample can be brought into sufficient contact with each other thermally, and that the temperature change of the heat medium during the warming process is so small that it can be ignored.

In the present embodiment, a plurality of units of the biological sample to be warmed may be disposed in respective different biological sample containers or may be collectively disposed in a single biological sample container. In a case where a plurality of units of the biological sample to be warmed are disposed in respective different biological sample containers, it is only necessary to take the biological sample containers from the place where they are stored, before use. However, in a case where a plurality of units of the biological samples are collectively disposed in a single biological sample container, the biological sample container is taken from the place where it is stored, and thereafter the units are transferred to respective different biological sample containers before use.

The biological sample container need only be capable of withstanding freezing temperature and low temperature, because the biological sample container may have disposed therein a sample to be frozen, a frozen sample, a sample preserved using a non-freezing, low-temperature preservation method, or the like. The biological sample container is, for example, preferably a container that can withstand −80° C., more preferably a container that can withstand −250° C. Specifically, the biological sample container is preferably a container made of a synthetic resin such as polyethylene, polypropylene, polystyrene, or polyethylene terephthalate.

The biological sample container is preferably hermetically closed, in order to prevent the biological sample in the biological sample container from being contaminated and prevent the biological sample from leaking out of the biological sample container and contaminating a worker or a work site. Since the biological sample container is put into the heat medium container which has the heat medium disposed therein, the biological sample container is preferably hermetically closed especially to prevent the heat medium from entering the biological sample container.

The biological sample container is disposed in the heat medium container such that the biological sample disposed in the biological sample container and the heat medium can exchange heat through the biological sample container. In order to allow the biological sample and the heat medium to be capable of exchanging heat, the biological sample container may be disposed in the heat medium container such that the external wall of the biological sample container and the heat medium at least partially physically contact each other. Specifically, it is preferable that the biological sample container be disposed in the heat medium container such that the biological sample container is at least partially immersed in the heat medium.

The biological sample container may be disposed in the heat medium container such that, assuming that the length of the tubular heat medium container is parallel to the vertical direction, the entire biological sample in the biological sample container is located lower than the surface of the heat medium in the vertical direction. This increases the area of thermal contact between the heat medium and the biological sample, making it possible to achieve more efficient heat transfer between the biological sample and the heat medium.

Note that, even if the external wall of the biological sample container and the heat medium are not in physical contact with each other at the point in time at which the biological sample container is put into the heat medium container, the more efficient heat transfer between the biological sample and the heat medium can still be achieved, provided that the external wall of the biological sample container and the heat medium physically contact with each other when the heat medium is caused to flow in the step of moving (described later).

In a case where the heat medium container includes a positioning part, the biological sample container may be disposed in the heat medium container such that the heat medium and the biological sample can exchange heat through the positioning part. With this configuration, the heat medium and the biological sample exchange heat, but the heat medium and the biological sample container do not directly contact each other. This makes it possible to reduce the risk that the heat medium will enter the biological sample container and contaminate the biological sample.

(Step of Closing)

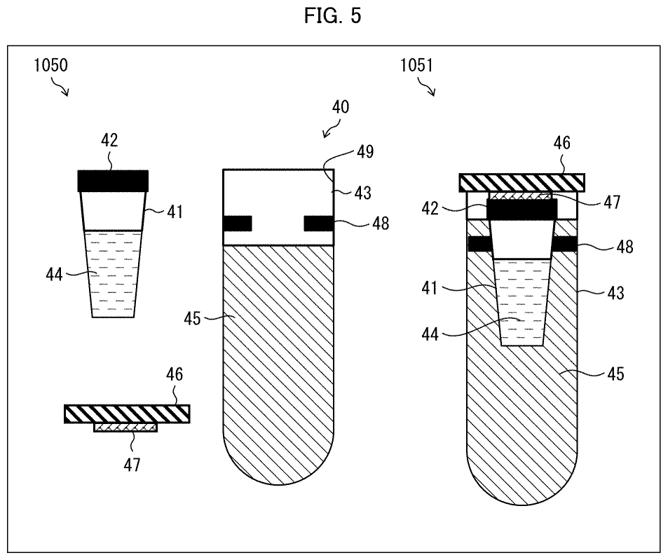

The step of closing involves closing the inlet through which the heat medium is injected (hereinafter “heat medium inlet”) of the heat medium container, in order to prevent the heat medium from leaking out of the heat medium container. The step of closing is preferably carried out after the step of putting. The heat medium inlet of the heat medium container can be closed by, for example, covering the heat medium inlet with a lid. Provided that the heat medium inlet of the heat medium container is closed, the heat medium and the biological sample container are prevented from going out through the heat medium inlet and prevented from contaminating a worker or a work site even when the heat medium container is moved in the step of moving (described later), even in the case of an embodiment in which the positioning part or the like does not have the function of blocking the heat medium from leaking out of the heat medium container (for example, see FIG. 5 described later) or in the case of an embodiment in which the positioning part itself is not present (for example, see FIG. 1).

Note, however, that there is no limitation on when the step of closing is carried out, provided that the objective (the heat medium does not leak out of the heat medium container) is achieved. For example, although the immediately preceding paragraph states that the step of closing is carried out after the step of putting, the step of putting can be carried out after the inlet of a heat medium container 2 (which is deformable as described earlier in the instance (2)) is closed in the step of closing.

The step of closing is not limited as to what member is used to close the inlet, provided that the above-described objective is achieved. For example, the resin film 11 in FIG. 2 functions as a position-fixing member which fixes the biological sample container 5 in place and also functions to prevent the heat medium 4 from leaking out of the heat medium container 2. Therefore, providing the inlet with a member that prevents the heat medium from leaking out of the heat medium container through the inlet before the step of moving is carried out (any time after the heat medium 4 is put into the heat medium container 2) corresponds to the step of closing. Note that the lid 6 in FIG. 2 closes the opening 3 and prevents the biological sample container 5 from going out of the heat medium container 2 during the step of moving (described later). The heat medium in the heat medium container does not leak through the pocket-shaped positioning part 3 into the space where the biological sample container is present, and therefore, needless to say, the heat medium does not leak out of the heat medium container 2 through the lid 6.

(Step of Moving)

In the step of moving, it is preferable that the heat medium be moved by moving the heat medium container with its heat medium inlet closed. In the step of moving, the heat medium is moved by moving the heat medium container having the biological sample container attached thereto so that the heat medium flows in the heat medium container and circulates in the heat medium container. This achieves more efficient heat exchange between the heat medium and the biological sample (through the biological sample container), making it possible to warm the biological sample in a shorter time.

Note, however, that the step of moving is not limited as to how it is carried out, provided that the objective (move the heat medium present in the heat medium container 2 having its inlet closed) is achieved. For example, a nozzle that supplies a fluid into the heat medium container 2 can partially close the inlet. Additionally or alternatively, for example, the heat medium container 2 having its inlet closed can have a fan therein. Both the nozzle and the fan (means to circulate the heat medium) cause circulation of the heat medium in the heat medium container 2. Provided that the nozzle or the fan is present, the step of moving does not need to involve moving the heat medium container 2.

The step of moving may be carried out with use of an apparatus that applies vibration to the heat medium container; however, it is preferable that the heat medium container be moved by holding and shaking the heat medium container by hand. Holding and shaking the heat medium container by hand is easier, because this does not necessitate using an apparatus that applies vibration. Especially in a case where cells to be warmed are warmed in an operating room where graft surgery is carried out, shaking by hand is more preferred because the above-mentioned apparatus is difficult to place in the operating room. Note that the apparatus that applies vibration to the entire heat medium container can be, for example, an apparatus known in this field (such as a tube rotator or a shaker). An example of a mechanism that circulates the heat medium without directly applying vibration to the heat medium container would be an embodiment in which a circulating means is provided, such as a nozzle that ejects a fluid into the warming container or a fan that circulates a fluid in the warming container and thereby achieves efficient heat transfer between the biological sample container and the heat medium.

The step of moving is carried out preferably under a condition in which the heat medium has a temperature of not lower than 20° C. and not higher than 40° C. This makes it possible to warm the biological sample in a shorter time with no or little damage to the biological sample. Furthermore, provided that the heat medium has a temperature of not lower than 20° C. and not higher than 40° C., warming is carried out at a temperature substantially the same as 37° C. which is usually employed in a conventional thawing method using a water bath. Note that the temperature of the heat medium here means the temperature before the heat medium exchanges heat with the biological sample.

The step of moving is carried out preferably under a condition in which the heat medium has a temperature of not lower than 20° C. and not higher than 27° C. With this configuration, the temperature of the heat medium is substantially the same as room temperature, and therefore it is not necessary to heat the heat medium. This eliminates the need for a heat source which is a heating element for heating the heat medium. Even in a case where the heat medium has a temperature lower than the above-described temperature range, the heat medium can be heated to the temperature range with body heat by holding the heat medium container by human hand.

In the step of moving, the heat medium container is preferably shaken for not shorter than 10 seconds and not longer than 600 seconds. When the time for which the heat medium container is shaken is within the above range, the heat medium efficiently flows within the heat medium container, and more efficient heat exchange between the heat medium and the biological sample is achieved. In the step of moving, the heat medium container is shaken for more preferably not shorter than 10 seconds and not longer than 300 seconds, even more preferably not shorter than 40 seconds and not longer than 180 seconds. In the step of moving, the heat medium container preferably continues to be moved until the biological sample has been warmed; however, the heat medium container may be moved intermittently. For example, the heat medium container may be moved for a certain period of time, allowed to stand, and then moved again.

In the step of moving, the heat medium container is shaken preferably at not less than 30 rpm and not more than 120 rpm. When the heat medium container is shaken at a rate within the above range, the heat medium efficiently flows within the heat medium container, and more efficient heat exchange between the heat medium and the biological sample is achieved.

In a case where the heat medium container is a tubular structure having opposite ends one of which is a closed end and the other of which is an open end, it is preferable, in the step of moving, that the heat medium container be shaken such that one of the opposite ends in the lengthwise direction of the tubular structure is fixed and the other of the opposite ends swings through an angle of not less than 90 degrees. Such a way of shaking the heat medium container is referred to as “mixing by inversion” in the present specification. By subjecting the heat medium container to the mixing by inversion, it is possible to force the heat medium to flow. This makes it possible to warm the biological sample in a short time even if the temperature of the heat medium is relatively low.

The angle of swinging movement during the mixing by inversion is more preferably not less than 120 degrees, even more preferably not less than 150 degrees. It is also preferable that the heat medium container be subjected to the mixing by inversion such that the opposite ends in the lengthwise direction of the tubular structure change places in the vertical direction at least once. Specifically, when the heat medium container is subjected to the mixing by inversion by holding one end of the heat medium container by human hand such that the length of the heat medium container is parallel to the vertical direction and swinging the other end horizontally, the other end is swung through a large angle so that the other end is located higher than the one end. This is preferred because the heat medium flows more greatly.

When the heat medium container is subjected to the mixing by inversion, the heat medium circulates to the extent that a forced flow of the heat medium occurs efficiently enough. Therefore, surprisingly, it is possible to warm the biological sample in a short time even if the temperature of the heat medium is low, e.g., 22° C. This temperature is lower than 37° C., which is typically employed in a conventional, general thawing method using a water bath or the like. Therefore, thermal damage caused on the biological sample is significantly less than the conventional method. Furthermore, since 22° C. is general room temperature, a heat source for heating the heat medium is not necessary in cases where the heat medium container is subjected to the mixing by inversion.

According to a conventional method using a water bath, a large amount of water on the order of 10 L is necessary as the heat medium. This is difficult to carry, routine cleaning is troublesome, and, if the routine cleaning is not done, germs thrive and cause unsanitary conditions. In contrast, a warming method in accordance with an aspect of the present invention requires only a very small amount of heat medium, e.g., not more than 50 mL. This is easy to carry, easy to dispose of after use, and routine cleaning is not necessary.

A conventional method of thawing a biological sample by heating the biological sample with a heater does not require a large amount of water and also the apparatus is small; however, the heater becomes hot temporarily, and therefore the biological sample is prone to damage. In addition, there are variations in performance between heaters used, and reproducibility of warming is not achieved in some cases. In contrast, according to a warming method in accordance with an aspect of the present invention, the biological sample can be warmed in a short time even though the temperature of the heat medium is low such as a temperature at or near room temperature. This causes no or little thermal damage to the biological sample, and eliminates the need for a heat source for heating the heat medium. Furthermore, warming is carried out by a very simple operation, i.e., the heat medium container having the heat medium disposed therein is held and shaken by human hand. This generates no or few variations among warming operations.

[Warming Container]

A warming container in accordance with another aspect of the present invention is a warming container for warming a biological sample, including: a heat medium container configured to have a heat medium disposed therein and have a biological sample container disposed therein, the biological sample container being configured to have a biological sample disposed therein; and a positioning part which is attached to the heat medium container and which is configured to keep the biological sample container in position.

A warming container in accordance with another preferred aspect of the present invention is a warming container for a biological sample, including: a biological sample container configured to have a biological sample disposed therein; a heat medium container configured to have a heat medium disposed therein; and a positioning part which is attached to the heat medium container and which is configured to keep the biological sample container in position. Note that the warming container can be regarded as, for example, a warming container for warming a biological sample (preferably a frozen or cooled biological sample) disposed in a biological sample container, the warming container including: a heat medium container configured to have disposed therein the biological sample container and a heat medium; and a positioning part which is provided inside the heat medium container and which is configured to keep the biological sample container in position. The following description will discuss the warming container in accordance with the preferred aspect of the present invention with reference to FIGS. 1 and 2.

As illustrated in FIG. 1, a warming container 1 includes a heat medium container 2. The warming container 1 is configured such that a biological sample container 5 is put into the heat medium container 2 and a biological sample disposed in the biological sample container 5 is warmed. A heat medium is put into the heat medium container 2, the biological sample container 5 is also put into the heat medium container 2, and the heat medium container 2 is closed with a lid 6.

The heat medium 4 is disposed in the heat medium container 2. The biological sample container 5 is put into the heat medium container 2 through an opening 3, and the biological sample in the biological sample container 5 and the heat medium 4 can exchange heat through the biological sample container 5. The opening 3 is closed with the lid 6 so that the heat medium 4 and the biological sample container 5 do not go out of the heat medium container 2 of the warming container 1.

The warming container further includes a resin film 11 which functions as a positioning part, like a warming container 10 illustrated in FIG. 2. The resin film 11 is in the form of a pouch (in the form of a pocket) so that the pouch or pocket can have the biological sample container 5 disposed therein. The resin film 11 is provided such that the resin film 11 covers the opening 3, that the top of the pouch is folded over the edge of the opening 3, and that the resin film 11 is in close contact with the heat medium container 2. That is, the resin film 11 in FIG. 2 functions as a position-fixing member that fixes the biological sample container 5 in place and also functions to prevent the heat medium 4 from leaking out of the heat medium container 2. For the resin film 11 to be fixed to the heat medium container 2, a parafilm or the like may be put around the portion of the resin film 11 folded over the edge of the opening 3. For convenience, the parafilm does not need to be wrapped tight; however, when firm fixation is required, the parafilm may be fixed with use of appropriate adhesion, wrapping, or the like which are known to those skilled in the art.

The heat medium container 2 need only be capable of having disposed therein the heat medium put into the heat medium container 2 through the opening 3. The heat medium used in the present invention does not become hot; therefore, the heat medium container 2 does not need to be heat resistant. Specifically, the heat medium container 2 is more preferably a container made of a synthetic resin such as polyethylene, polypropylene, polystyrene, or polyethylene terephthalate, or the like.

The heat medium container 2 is preferably a container that is easily disposable after use, which makes it possible to prevent cross-contamination. Furthermore, the heat medium container 2 is, for easy holding by human hand, preferably a tubular structure which has opposite ends one of which is a closed end and the other of which is an open end. It is more preferable that the tubular structure have, in a cross section perpendicular to the longitudinal direction of the tubular structure, a diameter of not less than 5 mm and not more than 200 mm. It is further preferable that the warming container can be carried by human hand. For example, a commercial centrifuge tube can be suitably used as the warming container.

Note that the heat medium container 2 does not need to be in the form of an axially symmetric centrifuge tube like those illustrated in FIGS. 1 and 2. Containers of various shapes such as chemical bottles, PET bottles, and the like can be suitably used. Note, however, that, in a case where the warming container does not have rigidity, e.g., in a case where the warming container is in the form of a pouch, the warming container is, for example, preferably inserted in a tubular structure in order that, when the heat medium is caused to circulate in the step of moving etc., the deformation of the warming container itself is minimized and the heat medium circulates within the warming container efficiently with good reproducibility.

The lid 6 need only cover the opening 3 and seal the heat medium container 2. A lid fitted into the opening 3, a threaded lid that opens and closes the opening 3 by rotation, or the like can be suitably used as the lid 6.

The heat medium 4 need only be capable of exchanging heat with the biological sample in the biological sample container 5. The heat medium 4 need only be a fluid having a heat capacity that can maintain a certain temperature for a certain period of time. The heat medium 4 is preferably at least one selected from the group consisting of water, isotonic solutions, and water which has an antibacterial agent dissolved therein. In a case where the biological sample is cells or a cell mass, the use of an isotonic solution as the heat medium 4 makes it possible to reduce the risk that the cells will be damaged even if the heat medium 4 and the biological sample contact each other.

The warming container in accordance with an aspect of the present invention can be suitably used in a warming method in accordance with an aspect of the present invention. Specifically, the warming container in accordance with an aspect of the present invention can be used in a warming method in accordance with an aspect of the present invention, and is capable of warming a biological sample easily and safely with no or little damage to the biological sample.

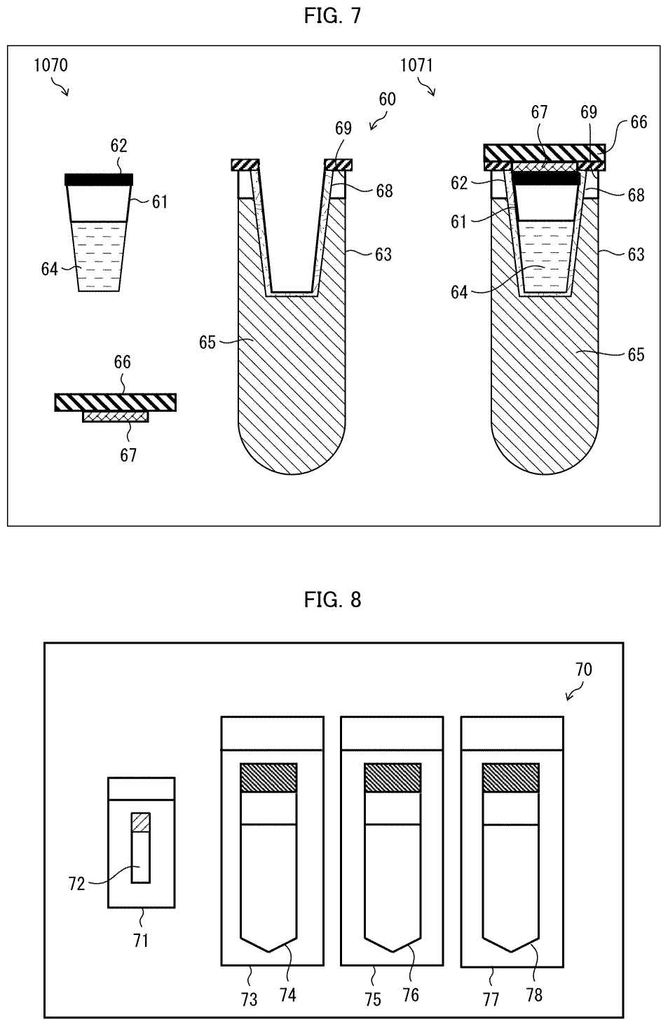

Furthermore, since the warming container in accordance with an aspect of the present invention includes a positioning part, the movement of the biological sample container within the heat medium container is restricted. This makes it possible to eliminate the likelihood that the biological sample container will be displaced greatly in the step of moving (described later) and hit the lid or the like and that the container will be broken. Furthermore, in the case of an embodiment in which, like FIGS. 2 and 7, the positioning part is in the form of a pocket or a container and is configured to prevent the heat medium and the biological sample container from directly contacting each other within the heat medium container, even in the event of surface cracking resulting from freezing or loosening of a cap that would frequently occur in the case of commercially-available cryogenic tubes, it is possible to reduce the risk that the heat medium will flow into the biological sample container and the biological sample will be contaminated.

The following description will discuss Specific Examples 1 to 3 of the foregoing instance (2) in detail. The heat medium container in the instance (2) can be a container made of a non-rigid (flexible) material. Such a container is excellent as a heat medium container in that the positioning part can be easily formed.

### Specific Example 1

A heat medium container of Specific Example 1 is, for example, a pouch made of a flexible material. A heat medium is enclosed in the pouch. The pouch is folded, at a certain position, around a biological sample container (the biological sample container is put into the space defined by the folded pouch such that the biological sample container is in contact with the outside surface of the pouch). The pouch is filled with a heat medium in advance, and the inlet of the pouch is closed in advance before making contact with the biological sample container. That is, the pouch is prepared by closing the inlet without having the biological sample container disposed therein. The pouch can be prepared by a method generally used in, for example, the process of processing a source material such as a film into a vinyl pouch or a plastic pouch or the process of closing a pouch filled with stuff; therefore, the pouch can be easily prepared and can be easily filled with a heat medium.

The pouch of Specific Example 1 may include a structure for attachment/fixation of the biological sample container to the outside surface of the pouch. The structure can be a string structure that forms a loop together with the outside surface or a sheet structure that forms a pocket together with the outside surface. The pouch of Specific Example 1, including the structure, is capable of more firmly pushing the biological sample container against the outside surface of the pouch. Such structures each serve as a positioning part.

The pouch can be further disposed in a guide member such as a hollow cylindrical container. The cylindrical container has a definite shape, and therefore easily maintains the pouch, which is easily deformable, in a certain folded state (it is easy to reproduce substantially the same state of contact between the outside surface of the pouch and the biological sample container). Since the cylindrical container restricts the deformation of the pouch, the circulation of the heat medium and the thermal contact between the heat medium and the biological sample container are not or little hindered by, for example, a change in shape of the pouch. It is therefore possible to efficiently transfer heat from the heat medium to the biological sample container. It is easy to apply vibration to a cylindrical container that has a definite shape instead of an indefinite pouch shape. It is preferable that the pouch further contain air therein. This is because the air inside the pouch serves to stir the heat medium when the pouch is moved. The cylindrical container can be made of any rigid material (such as paper, wood, resin, metal, or the like) in order to have a definite shape. Note that, although the hollow guide member is in the form of a cylinder in this example, the hollow guide member may be a member having a curved cross section other than a circle or a polygonal cross section.

### Specific Example 2

Another example of the pouch is a pouch which has a recess in the outside surface thereof. The recess has: an opening at the outside surface of the pouch; a hollow portion extending toward the inside of the pouch; and a bottom that is located opposite the opening and that closes the hollow portion. That is, the recess is so shaped as to accommodate the biological sample container. It is very easy to make a recess in a pouch which is made of a flexible material. The pouch of Specific Example 1 and the pouch of Specific Example 2 therefore have the same advantages except for the presence of the recess.

The pouch of Specific Example 2 is configured to have the biological sample container disposed in the recess thereof, and therefore does not need to be folded. It is possible to prevent the biological sample container from falling out of the recess of the pouch of Specific Example 2 simply by closing the opening of the recess or fixing the biological sample container from outside the recess. That is, the pouch of Specific Example 2 can achieve the same advantages as the pouch of Specific Example 1 even when the pouch of Specific Example 2 is not disposed in a guide member. The pouch of Specific Example 2 is therefore better than the pouch of Specific Example 1 in terms of handleability. Putting the pouch of Specific Example 2 in a guide member enhances the advantages of the pouch of Specific Example 2.

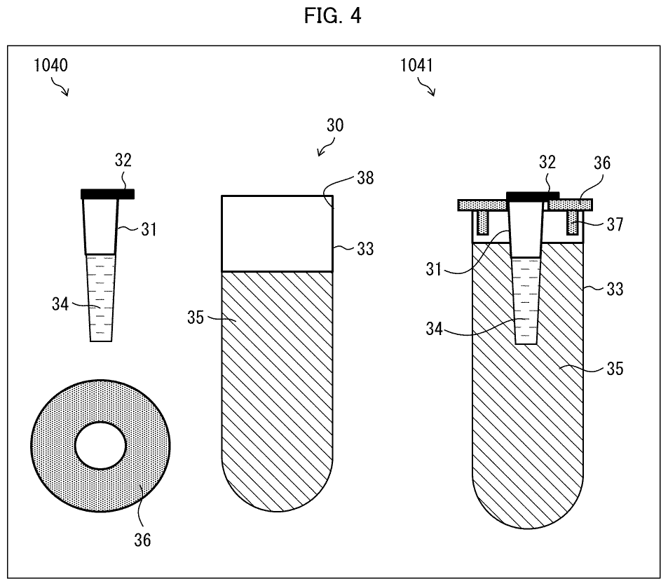

The following description will discuss other examples of a warming container in accordance with an aspect of the present invention, with reference to FIGS. 3 to 7. FIGS. 3 to 7 schematically illustrate warming containers in accordance with other aspects of the present invention.

<Variation 1>

1030 of FIG. 3 illustrates a warming container 20 and a biological sample container 21 which is not disposed in the warming container 20. 1031 of FIG. 3 illustrates the warming container 20 which has the biological sample container 21 disposed therein. As illustrated in 1030 of FIG. 3, a heat medium container 23 has a heat medium 25 disposed therein, and the biological sample container 21 has a biological sample 24 disposed therein. The biological sample container 21 has, on top of the opening thereof, a fixing member 22 which fixes the biological sample container 21 in place within the heat medium container 23.

The fixing member 22 has a portion that projects outward with respect to the outer periphery of the biological sample container 21. Therefore, when the biological sample container 21 is put into the heat medium container 23, the projection portion abuts the opening 26 of the warming container 20, prevents the biological sample container 21 from moving inward from that position, and thereby fixes the biological sample container in place within the heat medium container 23. The portion projecting outward with respect to the outer periphery of the biological sample container 21, of the fixing member 22, may be a structure in the form of a tab. The fixing member 22 may be provided on a lid that seals the biological sample container 21. Alternatively, the fixing member 22 itself may function as a lid for the biological sample container 21.

Since the biological sample container 21 is fixed by the fixing member 22 in place within the heat medium container 23, the movement of the biological sample container 21 within the heat medium container 23 is restricted when the warming container 20 is subjected to the step of moving such as shaking. This makes it possible to prevent the biological sample container 21 and the heat medium container 23 from mechanically hitting each other. It is also possible to eliminate the likelihood that the biological sample container 21 will rise by its buoyancy and that the thermal contact between the biological sample container 21 and the heat medium 25 will be hindered.

In Variation 1, the fixing member 22 functions also as a lid that closes the opening 26 of the warming container 20, as illustrated in 1031 of FIG. 3. The fixing member 22, in order to function as a lid for the warming container 20, has an outer diameter greater than that of the opening 26. Note that a lid for the warming container 20 may be separately provided. In a case where the step of moving such as shaking is not necessary during warming or in a case where the step of moving such as shaking to the extent that does not greatly change the surface of the heat medium 25 is enough for warming, e.g., in a case where the outer diameter of the biological sample container 21 is small and therefore heat is well conducted from the surface of the biological sample container 21 to the interior of the biological sample container 21, a lid for the warming container 20 does not need to be provided separately.

<Variation 2>

1040 of FIG. 4 illustrates a warming container 30 and a biological sample container 31 which is not disposed in the warming container 30. 1041 of FIG. 4 illustrates the warming container 30 which has the biological sample container 31 disposed therein. As illustrated in 1040 of FIG. 4, a heat medium container 33 has a heat medium 35 disposed therein, and the biological sample container 31 has a biological sample 34 disposed therein. The warming container 30 is different from the warming container 20 in that the warming container 30 includes a fixing member 36 in the form of a ring.

The biological sample container 31 is sealed with a lid 32, and at least part of the lid 32 projects outward with respect to the outer periphery of the biological sample container 31. The lid 32 is configured such that, when the biological sample container 31 is inserted in the hole in the middle of the fixing member 36, the projecting portion of the lid 32 abuts the fixing member 36, and prevents the biological sample container 31 from moving inward from that position. When the biological sample container 31 inserted in the fixing member 36 is put into the heat medium container 33, the fixing member 36 abuts an opening 38 of the warming container 30, and the biological sample container 31 is thereby fixed in place within the heat medium container 33. The fixing member 36 has an outer diameter greater than that of the opening 38 and functions as a lid for the warming container 30; however, a lid may be provided separately.

Since the biological sample container 31 is fixed by the fixing member 36 in place within the heat medium container 33, the movement of the biological sample container 31 within the heat medium container 33 is restricted when the warming container 30 is subjected to the step of moving such as shaking. This makes it possible to prevent the biological sample container 31 and the heat medium container 33 from mechanically hitting each other. It is also possible to eliminate the likelihood that the biological sample container 31 will rise by its buoyancy and that the thermal contact between the biological sample container 31 and the heat medium 35 will be hindered.

Variation 2 is advantageous in a case where, for example, a container with a small outer diameter, such as a microtube for holding a small amount of biological sample (e.g., template, primer, or protein extract), is used as the biological sample container. Note that, as illustrated in 1041 of FIG. 4, the fixing member 36 may have, at the bottom thereof, a protrusion 37 in the form of a circular tube. This makes it possible to prevent the fixing member 36 from slipping off the warming container 30.

<Variation 3>

1050 of FIG. 5 illustrates a warming container 40 and a biological sample container 41 which is not disposed in the warming container 40. 1051 of FIG. 5 illustrates the warming container 40 which has the biological sample container 41 disposed therein. As illustrated in 1050 of FIG. 5, a heat medium container 43 has a heat medium 45 disposed therein, and the biological sample container 41 has a biological sample 44 disposed therein and is sealed with a lid 42. The warming container 40 is different from the warming container 20 in that the heat medium container 43 has an abutment member 48 provided therein.

The abutment member 48 is located within the heat medium container 43 near an opening 49, and is configured such that the outside surface of the biological sample container 41 abuts the abutment member 48 and that the biological sample container 41 is prevented from moving inward from that position. The contact between the abutment member 48 and the outside surface of the biological sample container 41 may be point contact, line contact, or surface contact; however, it is preferable that the area of contact between the biological sample container 41 and the heat medium 45 be greater.

As illustrated in 1051 of FIG. 5, the biological sample container 41 is disposed in the heat medium container 43 such that the biological sample container 41 abuts the abutment member 48, and the warming container 40 is closed with the lid 46. The lid 46 has an abutment member 47 that abuts the biological sample container 41 when the warming container 40 is closed with the lid 46. When the warming container 40 is closed with the lid 46, the abutment member 47 abuts the biological sample container 41, and the lid 46 thereby pushes the biological sample container 41 down from above. This makes it possible to more stably fix the biological sample container 41 within the heat medium container 43. This makes it possible to prevent the biological sample container 41 and the heat medium container 43 from mechanically hitting each other. It is also possible to eliminate the likelihood that the biological sample container 41 will rise by its buoyancy and that the thermal contact between the biological sample container 41 and the heat medium 45 will be hindered.

<Variation 4>

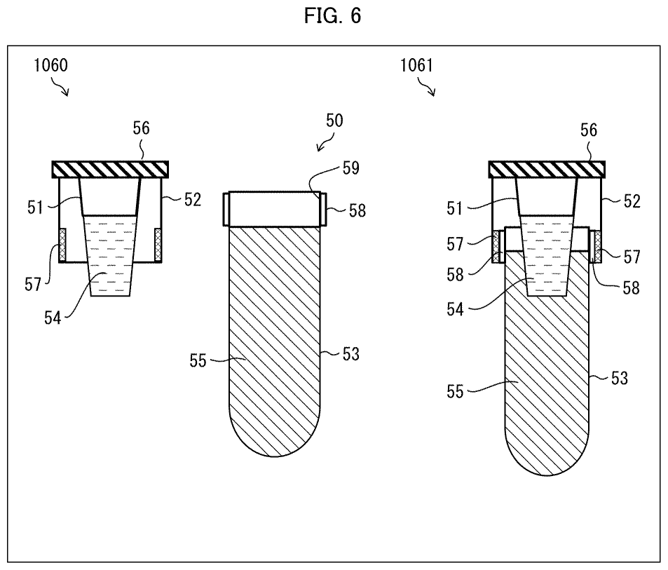

1060 of FIG. 6 illustrates a warming container 50 and a biological sample container 51 which is not disposed in the warming container 50. 1061 of FIG. 6 illustrates the warming container 50 which has the biological sample container 51 disposed therein. As illustrated in 1060 of FIG. 6, a heat medium container 53 has a heat medium 55 disposed therein, and the biological sample container 51 has a biological sample 54 disposed therein. The warming container 50 is different from the warming container 20 in that the warming container 50 includes a cover 52.

The biological sample container 51 is sealed with a lid 56, the lid 56 has a diameter greater than the diameter of the opening of the biological sample container 51, and the outer edge portion of the lid 56 projects outward with respect to the outer periphery of the biological sample container 51. The lid 56 has, on the side that contacts the biological sample container 51 and near the outer edge, a cover 52 in the form of a skirt that surrounds the sealed biological sample container 51. The cover 52 has a mating portion 57 on the inside surface at the open end. The heat medium container 53 has a mating portion 58 on the outside surface in the vicinity of the opening 59. The mating portion 57 and the mating portion 58 may be composed of, for example, threads that can fit each other.

The inner diameter of the cover 52 is greater than the outer diameter of the opening 59 by the thicknesses of the mating portion 57 and the mating portion 58. Therefore, when the biological sample container 51 is put into the heat medium container 53 through the opening 59 as illustrated in 1061 of FIG. 6, the cover 52 partially overlaps the heat medium container 53, the mating portion 57 is fitted into the mating portion 58, and the biological sample container 51 is fixed in place within the heat medium container 53. This makes it possible to prevent the biological sample container 51 and the heat medium container 53 from mechanically hitting each other. It is also possible to eliminate the likelihood that the biological sample container 51 will rise by its buoyancy and that the thermal contact between the biological sample container 51 and the heat medium 55 will be hindered.

<Variation 5>

1070 of FIG. 7 illustrates a warming container 60 and a biological sample container 61 which is not disposed in the warming container 60. 1071 of FIG. 7 illustrates the warming container 60 which has the biological sample container 61 disposed therein. As illustrated in 1070 of FIG. 7, a heat medium container 63 has a heat medium 65 disposed therein, and the biological sample container 61 has a biological sample 64 disposed therein. The warming container 60 is different from the warming container 20 in that the warming container 60 includes a positioning part 68.

The warming container 60 includes the positioning part 68 disposed such that the positioning part 68 covers an opening 69. The positioning part 68 is provided such that the positioning part 68 has the biological sample container 61 disposed in the space defined by the positioning part 68 and that the top of the positioning part 68 is hooked over the edge of the opening 69 of the heat medium container 63. The positioning part 68 is provided such that, when the biological sample container 61 is disposed in the space defined by the positioning part 68, a biological sample 64 and a heat medium 65 can exchange heat through the positioning part 68. Since the heat medium 65 does not enter the space defined by the positioning part 68, the biological sample container 61 and the heat medium 65 will not directly contact each other.

The inside surface of the positioning part 68 corresponds in shape to the outside surface of the biological sample container 61, and is configured such that, when the biological sample container 61 is disposed in the space defined by the positioning part 68, the inside surface of the positioning part 68 and the outside surface of the biological sample container 61 physically contact each other. The positioning part 68 is a rigid structure, and can be, for example, a metal container, a plastic container, or the like.

Note that, since the positioning part 68 is a rigid structure, the risk of breakage is low and cleaning is easy; however, the positioning part 68 does not closely fit the biological sample container 61 compared to the positioning part made of a resin film 11 illustrated in FIG. 2, and heat conducting property may decrease. One way to prevent such a reduction in heat conducting property would be to make the positioning part 68 from a highly thermally conductive material such as aluminum or to place another member that is composed of a highly thermally conductive, highly plastic material over the inside surface of the positioning part 68. The highly thermally conductive, highly plastic material can be, for example, a liquid ceramic coating such as “Cerac a (registered trademark)”. Using a commercially-available “Mazuharu Ichiban (registered trademark)” is easier.

The warming container 60 further includes a lid 66, and is configured such that the lid 66 closes the opening of the positioning part 68 disposed in the biological sample container 61 and thereby seals the warming container 60. The lid 66 has an abutment member 67 that is configured to be fitted into the opening of the positioning part 68 and abut the biological sample container 61 when the warming container 60 is sealed. When the warming container 60 is sealed with the lid 66, the abutment member 67 abuts the biological sample container 61 and the lid 66 thereby pushes the biological sample container 61 down from above. This makes it possible to prevent the biological sample container 61 from going out when the warming container 60 is subjected to the step of moving such as shaking and possible to cause the biological sample container 61 and the positioning part 68 to more firmly abut each other.

This makes it possible to prevent the biological sample container 61 and the medium holding container 63 from mechanically hitting each other. It is also possible to eliminate the likelihood that the biological sample container 61 will rise by its buoyancy and that the thermal contact between the biological sample container 61 and the heat medium 65 will be hindered. Furthermore, since the heat medium 65 and the biological sample container 61 do not directly contact each other, it is possible to reduce the risk that the heat medium 65 will enter the biological sample container 61 and the biological sample 64 will be contaminated.

[Kit for Warming]

A kit for warming a biological sample, in accordance with an aspect of the present invention, includes a heat medium container configured to have a heat medium and a biological sample container disposed therein. The heat medium container includes a positioning part configured to keep the biological sample container in position, and the biological sample container is configured to have a biological sample disposed therein.

A kit for warming a biological sample, in accordance with a preferred aspect of the present invention, includes: a warming container configured to have a heat medium disposed therein; and a biological sample container which is configured to have a biological sample disposed therein and which is capable of being disposed in the warming container. The kit for warming a biological sample may further include a wash liquid for use in washing the biological material. Additionally or alternatively, the kit for warming a biological sample may include a liquid for preservation of the washed biological sample.

The following description will discuss a kit in accordance with an aspect of the present invention with reference to FIG. 8. FIG. 8 schematically illustrates a kit for warming a biological sample (hereinafter “warming kit”) in accordance with an aspect of the present invention. A warming kit 70 is an example of a packaged kit that is used to carry a regenerative medicine product into a clean environment such as a hospital room or an operating room. As illustrated in FIG. 8, the warming kit 70 includes a set comprised of: a biological sample container package 71 which has a biological sample container 72 enclosed therein; a warming container package 73 which has a warming container 74 enclosed therein; a washing container package 75 which has a washing container 76 enclosed therein; and a storage container package 77 which has a storage container 78 enclosed therein.

The biological sample container 72 may have a biological sample disposed therein, and the biological sample container 72 may have been sterilized and disposed in a hermetically sealed container. The biological sample container 72 can be a biological sample container for use in the foregoing warming method in accordance with an aspect of the present invention. The warming container 74 has a heat medium disposed therein. The warming container can be the foregoing warming container in accordance with an aspect of the present invention. The washing container 76 has, disposed therein, a wash liquid for use in washing a warmed biological sample. The wash liquid can be, for example, an isotonic solution such as lactated Ringer's solution or PBS. The storage container 78 has, disposed therein, a liquid for preservation of a washed biological sample. The liquid for preservation can be, for example, lactated Ringer's solution or PBS. The washing container and the storage container may each be, for example, a commercially-available centrifuge tube having a lid attached thereto.

With regard to the packages, the biological sample container 72, which has a biological sample disposed therein, cannot be γ-ray-sterilized; however, the warming container package 73 having the warming container 74 enclosed therein, the washing container package 75 having the washing container 76 enclosed therein, and the storage container package 77 having the storage container 78 enclosed therein have preferably been sterilized by, for example, γ-ray sterilization. In a case where the biological sample is tissue, a cell mass, or the like, the warming kit 70 may include a holding means such as forceps to remove the biological sample from the biological sample container 72. Such a holding means preferably has also been packaged and γ-ray-sterilized. Furthermore, in the kit 70, the biological sample container 72 may have disposed therein a biological sample preserved at low temperature, and the kit 70 may further include a cold keeper configured to keep the biological sample in a cold state. The cold keeper may be a cold-keeping mechanism or a cold-keeping agent. Alternatively, the biological sample container 72 having a biological sample disposed therein may be disposed in a cold-keeping agent.

The following description will discuss a method of using the warming kit 70, based on an example in which the biological sample is mesenchymal stem cells (MSCs). After the warming kit 70 is carried into an environment where the warming kit 70 is to be used, such as an operating room, the biological sample container 72 having been taken out of the biological sample container package 71 by opening the biological sample container package 71 is put into the warming container 74 having been taken out of the warming container package 73 by opening the warming container package 73. Next, the warming container 74 having the biological sample container 72 disposed therein is moved and thereby the MSCs are quickly warmed, and the MSCs are thawed sufficiently. The warmed MSCs are transferred into the washing container 76 having been taken out of the washing container package 75 by opening the washing container package 75, and washed. Then, the washed MSCs are transferred into the storage container 78 having been taken out of the storage container package 77 by opening the storage container package 77, and stored until just before grafting.

The present invention is not limited to the embodiments, but can be altered by a skilled person in the art within the scope of the claims. The present invention also encompasses, in its technical scope, any embodiment derived by combining technical means disclosed in differing embodiments. Further, it is possible to form a new technical feature by combining the technical means disclosed in the respective embodiments.

### EXAMPLES

The following description will discuss Examples of the present invention.

[1-1: Evaluation of Conditions Under which Warming is Carried Out]

Conditions under which warming is carried out by a warming method in accordance with an aspect of the present invention were evaluated in a simulated environment. For convenience of description, a warming method of the present invention is referred to as “Tube-in-Tube method”. In the simulated environment, a frozen sample obtained by freezing 1 mL of a cryopreservation solution, instead of cells, was used as a biological sample, and conditions under which warming was carried out were evaluated. The cryopreservation solution had the following composition: 90% (v/v) STK (registered trademark) 2 (cytokine-free)+10% (v/v) DMSO (Wako 031-24051). A centrifuge tube with no positioning part was used as a warming container. 1 mL of the cryopreservation solution was put into a cryogenic vessel (Cryogenic Vial: WHEATON, Cat. W985865) serving as a biological sample container, and was frozen under a deep freezer at −80° C. The cryogenic vessel was put into a centrifuge tube (50 mL centrifuge tube: SUMITOMO BAKELITE CO., LTD. Cat. MS-56501) having disposed therein a heat medium in an amount shown in Table 1. The heat medium had a temperature shown in Table 1. The heat medium was circulated by a method shown in Table 1, and the biological sample in the cryogenic vessel was warmed and thawed.

For comparison, thawing was carried out by a water bath method using a 37° C. water bath. Note that the thawing using a 37° C. water bath is one of the conventional thawing methods which showed the most favorable result in the study conducted by the inventors of the present invention.

Table 1 shows conditions under which the frozen sample was thawed and the results of the thawing (time taken for thawing) obtained under the respective conditions.

TABLE 1

| | | | | | | Ratio of | |
|---|---|---|---|---|---|---|---|
| | | | | | | time | |
| | | | | | | taken for | |
| | Method | Temperature | | Circulation | Time | thawing to | |
| | of | of heat | Amount of | of heat | taken for | that of | |
| | thawing | medium | heat medium | medium | thawing | Condition 0 | |
| Condition | Water | 37° C. | Amount | Circulation | 140 | 1.0 | time |
| 0 | bath | | provided by | pump | seconds | | |
| | method | | manufacturer | contained in | | | |
| | | | | apparatus | | | |
| Condition | Tube-in- | 37° C. | 10 mL | Allowed to | 220 | 1.6 | times |
| 1 | Tube | | | stand | seconds | | |
| | method | | | | | | |
| Condition | Tube-in- | | | | | | |
| 2 | Tube | 37° C. | 10 mL | Shaking | 140 | 1.0 | time |
| | method | | | | seconds | | |
| Condition | Tube-in- | | | | | | |
| 3 | Tube | 24° C. | 10 mL | Shaking | 210 | 1.5 | times |
| | method | | | | seconds | | |
| Condition | Tube-in- | 24° C. | 40 mL | Mixing by | 160 | 1.1 | times |

| | | | |
|---|---|---|---|
| 4 | Tube | inversion | seconds |
| | method | | |

First, Condition 0 and Conditions 1 and 2 were compared. In the case of the Tube-in-Tube method at 37° C. in which the heat medium was allowed to stand without shaking, the time taken for thawing was 1.6 times that in the case of the water bath method at 37° C. On the contrary, when the heat medium was shaken to circulate the heat medium (Condition 2), the time taken for thawing was equivalent to that in the case of the water bath method. These results showed that, even in a case of a simple configuration like a warming container in accordance with an aspect of the present invention, thawing can be carried out as quickly as in the case of using the conventional water bath by shaking the warming container and circulating the heat medium.

With regard to Condition 3 in which the temperature of the heat medium was about room temperature (24° C.) and the heat medium was shaken, the time taken for thawing was 1.5 times that in the case of Condition 0 and Condition 2. To find out a reason therefor, a theoretical calculation was carried out on the temperature change of the heat medium based on the assumption that there is no heat exchange with an external environment similarly to the order estimation discussed earlier in an embodiment. It was found that, in theory, the temperature of the heat medium decreases by as much as 13° C. as the thawing proceeds. That is, it is considered that, in a case where the temperature of the heat medium is 24° C., it is necessary that the heat capacity of the heat medium be sufficient.

On the basis of the above results, the amount of the heat medium was tentatively increased in order to reduce the temperature change of the heat medium. An increase in amount of the heat medium not only results in an increase in heat capacity but also results in sufficient thermal contact between the circulating heat medium and the frozen sample. Thawing was carried out under Condition 4 in which, in addition to an increase in amount of the heat medium, the circulation method for applying movement to the heat medium was changed from simple “shaking” to stronger “mixing by inversion” in order to achieve a stronger forced heat transfer between the heat medium and the frozen sample.

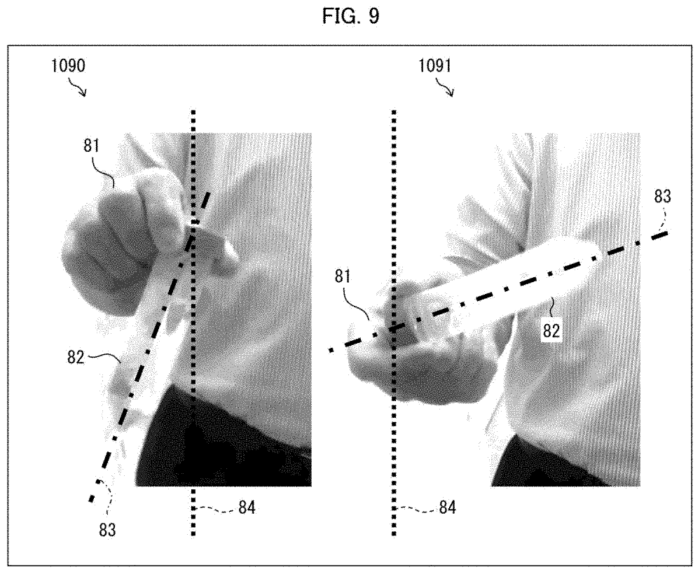

The following description discusses the mixing by inversion to which a warming container is subjected, with reference to FIG. 9. FIG. 9 shows an example of a method of mixing by inversion, and shows how a centrifuge tube with no cryogenic vessel inserted therein is subjected to mixing by inversion. Since FIG. 9 shows an example action, the operator in FIG. 9 has not taken a necessary measure such as wearing of gloves; however, the operator preferably takes necessary protective/contamination control measures in clinical practice or the like.

As shown in 1090 and 1091 of FIG. 9, a centrifuge tube 82 is subjected to mixing by inversion by holding the centrifuge tube 82 by human hand 81 and twisting the wrist. 1090 of FIG. 9 shows the centrifuge tube 82 with its bottom end fully pivoted counterclockwise, and 1091 of FIG. 9 shows the centrifuge tube 82 with its bottom end fully pivoted clockwise. In this example, a cycle consisting of (i) transition from the state shown in 1090 of FIG. 9 to the state in 1091 of FIG. 9 counterclockwise and (ii) transition from the state shown in 1091 of FIG. 9 to the state shown in 1090 of FIG. 9 clockwise was carried out successively and periodically at a rate of about 60 cycles per minute (60 rpm).

Note that, in 1090 and 1091 of FIG. 9, dot-dash line 83 representing the central axis of the centrifuge tube 82 and dashed line 84 representing the vertical direction are annotations provided for convenience of description, and these are not real constituent elements. The angle between the dashed line 84 and the dot-dash line 83 (assuming that the counterclockwise direction is “positive” direction in angular dimension) is about −30° in 1090 of FIG. 9 and about 120° in 1091 of FIG. 9.

That is, with a focus on the central axis of the centrifuge tube, the centrifuge tube is swung between about −30° and 120°, and swung through about 150°. In Condition 4 in which the mixing by inversion was carried out such that the centrifuge tube was swung through such a large angle, the time taken for thawing was substantially the same (1.1 times) as that in the case of the water bath method at 37° C. (Condition 1).

These results show that, according to the Tube-in-Tube method, even if the temperature of a heat medium is room temperature, it is possible to achieve the thawing speed substantially the same as in the case of a conventional method using a 37° C. water bath, by using a sufficient amount of heat medium and carrying out mixing by inversion that can cause a sufficient forced heat transfer.

[1-2: Warming with Use of Warming Container Provided with Positioning Part]

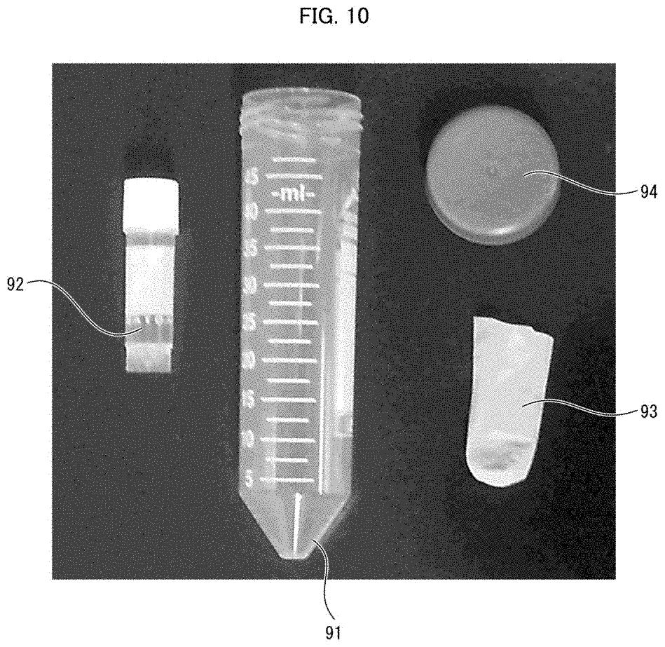

A warming method in accordance with an aspect of the present invention was evaluated in a simulated environment, with use of a warming container provided with a positioning part. First, a warming container provided with a positioning part was prepared in the following manner. FIG. 10 shows examples of a heat medium container and a biological sample container. As shown in FIG. 10, a tube 91 serving as a heat medium container, a resin film 93 configured to form a positioning part, a lid 94, and a cryogenic vessel 92 serving as a biological sample container were used. Note that a 50 mL centrifuge tube was used as the tube 91.

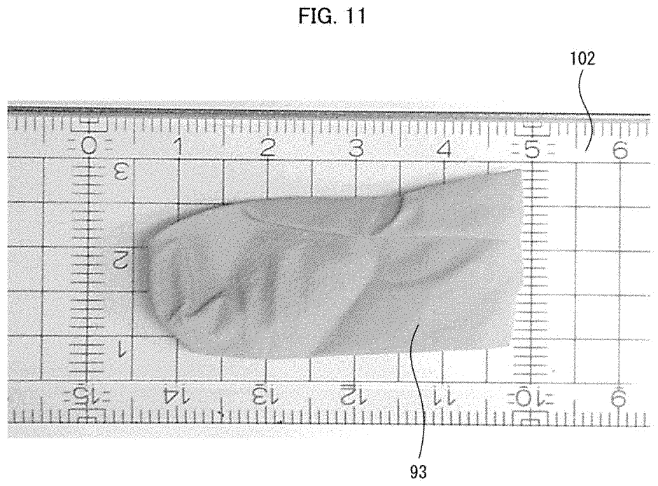

The resin film 93 is in the form of a pouch, and is sized such that pouch has the cryogenic vessel 92 disposed therein and that the pouch fits the cryogenic vessel 92. Specific dimensions of the resin film 93 are discussed with reference to FIG. 11. As is apparent from a comparison with a ruler 102 (0.5 cm graduated ruler), the resin film 93 has a length of about 4.5 cm and a width of about 1.5 cm. The resin film 93 was prepared by cutting off a part of a finger of a rubber glove for experiments called Lavender Nitrile (registered trademark).

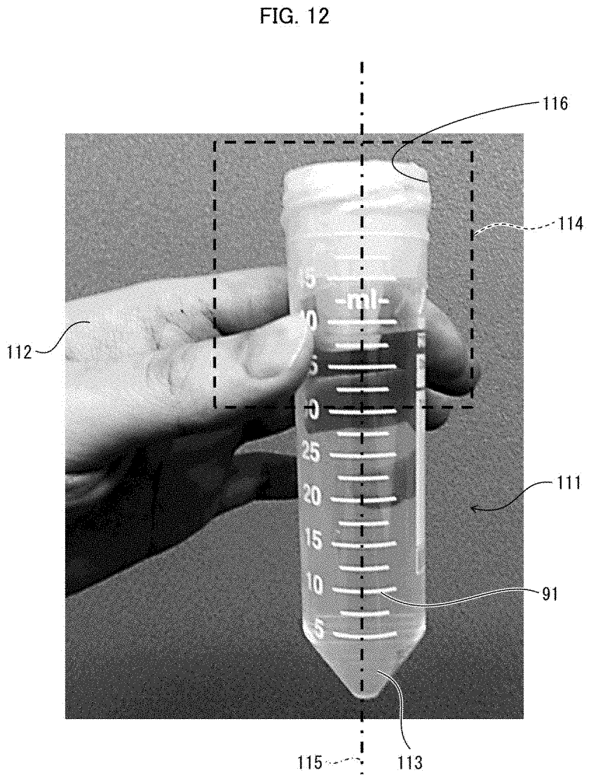

Next, a warming container 111 was prepared as shown in FIGS. 12 to 14. FIG. 12 shows the warming container 111 held by human hand 112. The tube 91 is symmetrical with respect to dot-dash line 115 representing the central axis of the tube 91, but may be a tube which is not axially symmetric. Note that the dot-dash line 115 is an annotation provided for convenience of description. The tube 91 has an opening 116 and a bottom 113. Note that, although a graduated tube was used, a tube not graduated may be used.

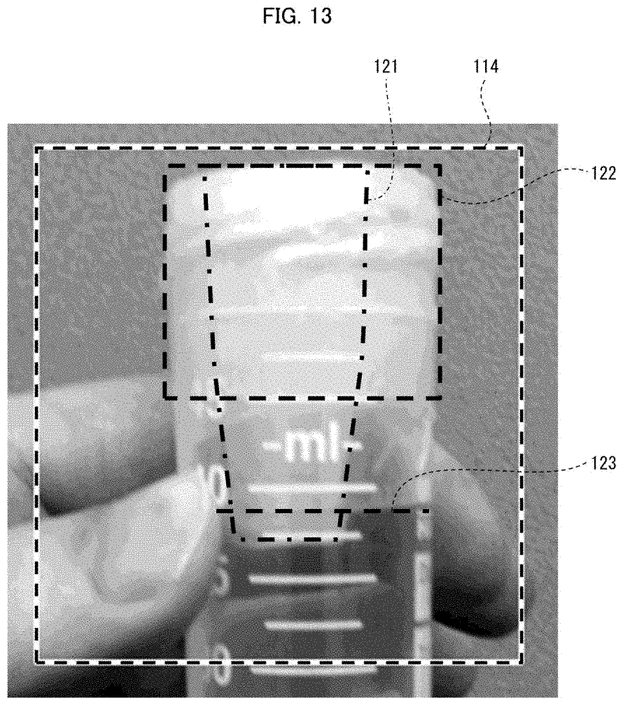

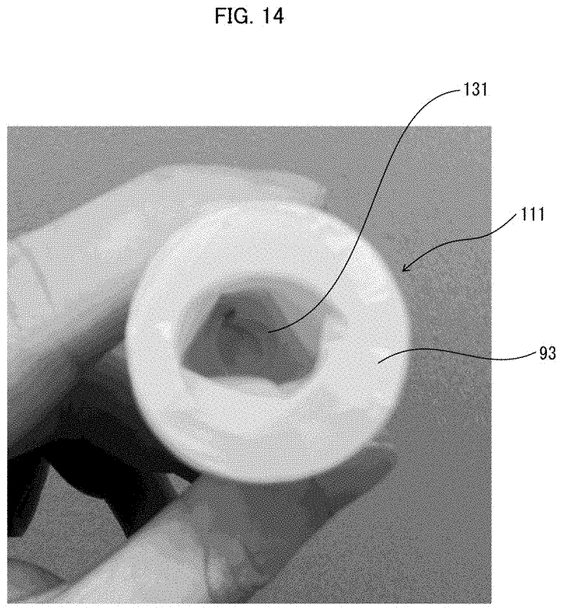

FIG. 13 is an enlarged view of the area enclosed by dashed line 114 of FIG. 12. The tube 91 has about 40 mL of a heat medium disposed therein, and the surface of the heat medium is represented by dashed line 123. The resin film 93 was attached to the opening 116 of the tube 91 by expanding the opening of the pouch and folding it over the edge of the opening 116. A parafilm was put around the area enclosed by dashed line 122 and the resin film 93 was fixed. The location of the resin film 93 attached to the opening 116 is represented by dot-dash line 121. The dot-dash line 121 and the dashed line 123 are annotations provided for convenience of description. In a top view of the opening 116 which has the resin film 93 attached thereto (FIG. 14), there is a pocket 131. The pocket 131 is configured to have the cryogenic vessel 92 inserted therein. The resin film 93 is fixed such that the resin film 93 forms a pocket.

A condition under which a biological sample is warmed was studied with use of the warming container 111 prepared as described above. Note, here, that a warming method in accordance with the present invention using the warming container 111 is referred to as “non-contact Tube-in-Tube method”. First, under the same condition as Condition 4 of Table 1, a biological sample was warmed and thawed by a non-contact Tube-in-Tube method with use of the warming container 111. There were variations in time taken for thawing (170 seconds to 220 seconds). Such time taken for thawing is 1.1 to 1.4 times the time taken for thawing under Condition 4 of Table 1, and is longer than the time taken for thawing under Condition 4 of Table 1. There was about one minute difference between the longest and shortest times, which was also an issue. It was thus found that there is room for improvement in the reproducibility of thawing characteristics. Further study was conducted in view of the above, and it was found that the time taken for thawing increases when, for example, there is an air space between the resin film 93 and the cryogenic vessel 92 or when the resin film 93 is sagging.

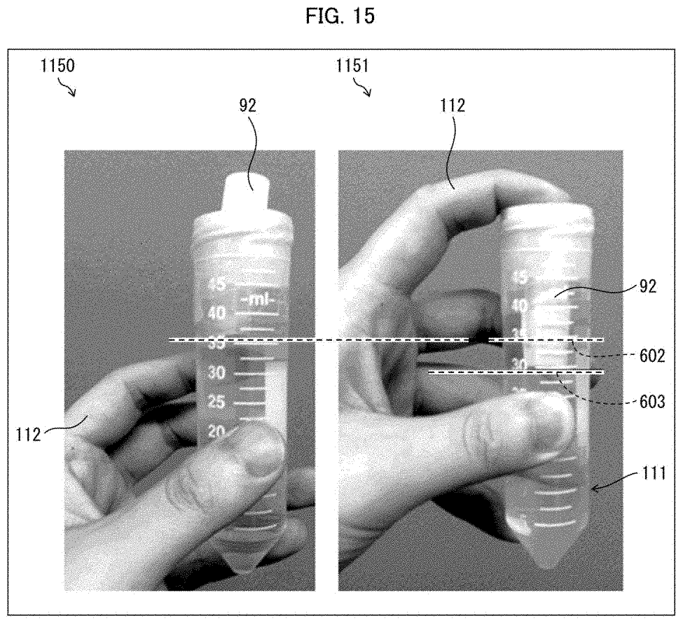

Diligent study was conducted on the basis of the above results, and it was found that, when the tension of the resin film 93 is adjusted by pushing the cryogenic vessel 92 into the pocket of the resin film 93 by about 1 cm as shown in FIG. 15, the cryogenic vessel 92 and the resin film 93 are brought into closer contact with each other, the heat exchanging property between the heat medium and the frozen sample improves, and therefore the time taken for thawing decreases. 1150 of FIG. 15 shows the cryogenic vessel 92 which has not yet been pushed into the space defined by the resin film 93, and 1151 of FIG. 15 shows the cryogenic vessel 92 which is being pushed in the space defined by the resin film 93. Note that the cryogenic vessel 92 was pushed into the space defined by the resin film 93 by human hand 112.

Before the cryogenic vessel 92 was pushed into the space defined by the resin film 93, the bottom end of the resin film 93 was located at the position represented by dashed line 602. After the cryogenic vessel 92 was pushed into the space defined by the resin film 93 by the human hand 112, the bottom end of the resin film 93 was located at the position represented by dashed line 603. That is, the cryogenic vessel 92 was pushed into the space defined by the resin film 93 by about 1 cm. Since the cryogenic vessel 92 was pushed into the space defined by the resin film 93 against the elastic resistance of the resin film 93 in such a manner, the cryogenic vessel 92 and the resin film 93 were brought into closer contact with each other. While the cryogenic vessel 92 was in this state, the biological sample was thawed by means of mixing by inversion. As a result, even though the temperature of the heat medium was 22° C., the time taken for thawing was equivalent to that in the case of using a 37° C. water bath.

Note that 1151 of FIG. 15 shows an example in which the cryogenic vessel 92 is pushed into the space defined by the resin film 93 by the human hand 112 so that the amount by which the cryogenic vessel 92 is pushed is easily recognizable; however, in practice, the cryogenic vessel 92 was pushed into the space defined by the resin film 93 by pushing the cryogenic vessel 92 with the lid 94. Since FIG. 15 shows an example action, the operator in FIG. 15 has not taken a necessary protective measure such as wearing of gloves; however, the operator preferably takes necessary protective/contamination control measures in clinical practice or the like.

[2-1: Establishment of Mesenchymal Stem Cells (MSCs) from Synovial Tissue]

Synovial tissue left over from an anterior cruciate ligament reconstruction surgery or the like was provided from a medical institution, through an appropriate ethical review and with patient's consent. The wet weight of the provided synovial tissue was measured, the synovial tissue was transferred into a centrifuge tube having placed therein 10 mL of gentamicin (Nichi-Iko Pharmaceutical)-containing DMEM (SIGMA), and washed. The synovial tissue was transferred to another centrifuge tube having placed therein 10 mL of gentamicin-containing DMEM, washed again, and then taken from the centrifuge tube onto a container. The washed synovial tissue was cut on the container into tissue fragments of not greater than 5 mm with use of sterilized scissors, suspended in gentamicin-containing DMEM, and then synovial tissue fragments were collected in a 50 mL centrifuge tube. Then, centrifugation was carried out at room temperature at 1500 rpm for 5 minutes, and a supernatant was removed.

STK (registered trademark) 1 (serum-free culture medium for primary MSC establishment, DS Pharma Biomedical Co., Ltd.) was added, the tissue fragments were seeded onto a 150 cm² dish (SUMITOMO BAKELITE CO., LTD.) at a seeding density of 2.5 mg (synovial tissue fragments)/cm² (surface area of culture plate), and cultured under a condition in which CO₂ concentration was 5% and temperature was 37° C. for 14 days (culture medium was changed on day 5, day 8, and day 11).

[1-2: Subculture of Synovial MSCs]

The proliferated MSCs were washed once with phosphate buffered saline (PBS, calcium-free, magnesium-free, PBS(−), Cell Science & Technology Institute, Inc.), and then detached with use of a cell detachment agent TrypLE Select CTS (Thermo Fisher Scientific Inc.), collected, and suspended in a washing medium (DMEM, Sigma). Then, the MSCs were transferred into a tube for centrifugation, subjected to centrifugation at 1500 rpm at room temperature for 5 minutes and were pellet down, and then a supernatant was removed.

The pellet down cells from the single-cell suspension (cell suspension) were again suspended in a washing medium, and the number of cells was counted using trypan blue staining. Next, the cells were seeded into STK (registered trademark) 2 in a 150 cm² dish (SUMITOMO BAKELITE CO., LTD.) at a density of 5000 cells/cm², cultured under a condition in which CO₂ concentration was 5% and temperature was 37° C. for 5 days (culture medium was changed on day 3), and the same operation was repeated until 3rd generation was obtained.

[2-3: Preservation of Intermediate Product]

The proliferated MSCs (3rd generation: P3) were washed once with PBS(−), were detached from the dish with use of a cell detachment agent TrypLE Select CTS and collected, suspended in DMEM, transferred into a tube for centrifugation, and then subjected to centrifugation at 1500 rpm at room temperature for 5 minutes and pellet down. The pellet down cells in a single cell state were again suspended in a washing medium, and the number of the cells was counted using trypan blue staining.

The cell suspension was again subjected to centrifugation at 1500 rpm at room temperature for 5 minutes and pellet down, a supernatant was removed, and then the pellet down cells were suspended in CELLBANKER2 (Nippon Zenyaku Kogyo Co., Ltd.) and cryopreserved in a deep freezer (in a −150° C. environment). Before the MSCs were used again, the MSCs were thawed with use of a 37° C. water bath for 2.5 minutes, washed with DMEM (thawed MSCs were transferred to a 15 mL tube having placed therein 10 mL of DMEM and subjected to centrifugation and pellet down, and a supernatant was removed), seeded into a STK (registered trademark) 2 in a 150 cm² dish at 5000 cells/cm², and cultured under a condition in which CO₂ concentration was 5% and temperature was 37° C. for 5 days.

[2-4: Preparation of gMSC (Registered Trademark) 1]

MSCs (5th generation: P5) were washed once with PBS(−), thereafter detached with use of a cell detachment agent TrypLE Select CTS, collected, suspended in DMEM, and then transferred to a tube for centrifugation. The suspension was again subjected to centrifugation at 1500 rpm at room temperature for 5 minutes, pellet down, a supernatant was removed, and the obtained cells were suspended in DMEM. The suspension was again subjected to centrifugation at 1500 rpm at room temperature for 5 minutes, pellet down, a supernatant was removed, the obtained cells were suspended in DMEM, and then passed through a cell strainer to remove aggregated cells and obtain a single-cell suspension. The suspension was further subjected to centrifugation at 1500 rpm at room temperature for 5 minutes and pellet down, a supernatant was removed, the obtained cells were suspended in STK (registered trademark) 2, and the number of the cells was counted using trypan blue staining.

The cells were seeded into STK (registered trademark) 2 in a 6 well plate (SUMITOMO BAKELITE CO., LTD.) at high density, i.e., a seeding density of 40×10⁴ cells/cm². That is, the number of cells per piece of MSC at the time of high-density seeding is 368 million. The cells were cultured in a 37° C., 5% CO₂ incubator for 7 days. The culture medium was changed on day 3 and day 5.

On day 7, tissue was mechanically detached from the culture plate, hung from the tip of Pipetman (registered trademark) and was crumpled. In this way, a cell mass of gMSC (registered trademark) 1 which is a scaffold-free three-dimensional structure was obtained.

[2-5: Measuring the Number of Cells in gMSC (Registered Trademark) 1]

The gMSC (registered trademark) 1 in the 6 well plate was washed twice with PBS(−), was then transferred into a 15 mL centrifuge tube having placed therein 1 mL of a 280 U/ml collagenase/50% TrypLE select solution, and digested at 37° C. Mixing by inversion was carried out ten times at 10-minute intervals, digestion was allowed to proceed until masses completely disappeared, and the total number of cells and the number of living cells were measured using trypan blue staining. The composition of the 280 U/ml collagenase/50% TrypLE select solution is as follows.

Collagenase: Worthington biochemical corporation, Cat. LS004154 Lot: 44D14883,
TrypLE select: Thermo Fisher Scientific Inc., Cat. A12859-01 Lot: 1905779,
DMEM: Sigma, Cat. D6046, Lot: RNBG0276

[2-6: Freezing gMSC (Registered Trademark) 1]

The gMSC (registered trademark) 1 in the 6 well plate was washed twice with PBS(−), was then transferred into a 2 mL cryogenic vial having placed therein 1 mL of a cryopreservation solution, and allowed to spontaneously freeze at −80° C. The composition of the cryopreservation solution is: 90% (v/v) STK (registered trademark) 2 (cytokine-free)+10% (v/v) DMSO (Wako 031-24051).

[2-7: Thawing gMSC (Registered Trademark) 1 and Measuring the Number of Cells]

After one-week preservation at −80° C., the cryogenic vial was removed from the freezer, and subjected to a warming experiment (described later). The gMSC (registered trademark) 1 which had thawed by warming was washed with DMEM, digested with a 280 U/ml collagenase/50% TrypLE select solution, and the total number of cells and the number of living cells were measured using trypan blue staining.

[2-8: Evaluation of Ability of Cells to Proliferate Via Re-Seeding of gMSC (Registered Trademark) 1]

The gMSC (registered trademark) 1 in a single cell state, obtained in the foregoing section 2-5, was washed twice with DMEM, and then re-seeded into a 6 well plate at 5,000 cells/cm². The total number of cells and the number of living cells were measured on day 5.

[3-1: Comparison Between Tube-in-Tube Method and Conventional Method]

For evaluation of the effects of the Tube-in-Tube method (not “non-contact”), pieces of gMSC (registered trademark) 1 prepared and cryopreserved by the same method were warmed by different warming methods for comparison of cell performance. In this example, thawing was carried out by the following warming methods: Tube-in-Tube method (see “thawing method B” below); 37° C. water bath method (see “thawing method A” below); and 37° C. heat block method (see “thawing method C” below). Note that the 37° C. heat block method is a method involving carrying out thawing with use of a heat block apparatus manufactured by STREX Inc. (Model No. SY-1) at a set temperature of 37° C. Note that, since gMSC (registered trademark) 1 is a three-dimensional cell mass, for measuring the number of cells, it is necessary to digest and decompose the cell mass until a single-cell suspension is obtained, as stated in the foregoing section 2-5. Therefore, a group whose number of cells was counted in accordance with the foregoing section 2-5 without carrying out a freezing operation (see “non-frozen group” below) was prepared as a control group which means “cells before freezing”. The details of the experiment are as follows.

(Preparation and Grouping of gMSC (Registered Trademark) 1)

Three strains of MSC (strain 1, strain 2, and strain 3) established based on the foregoing section 2-1 (different strains are from different donors) were each processed into gMSC (registered trademark) 1 in accordance with the methods stated in the foregoing sections 2-2 to 2-4. The gMSC (registered trademark) 1 derived from each strain was grouped into four groups consisting of non-frozen group, thawed-by-method-A group, thawed-by-method-B group, and thawed-by-method-C group (i.e., three strains×four groups=twelve groups in total). The sample size in each group was as follows: non-frozen group (N=3); thawed-by-method-A group (N=3); thawed-by-method-B group (N=3); and thawed-by-method-C group (N=3).

Each gMSC (registered trademark) 1 in the non-frozen group was digested and decomposed in accordance with the foregoing section 2-5 until a single-cell suspension was obtained, and the number of cells was measured under the condition of the section 2-5.

(Freezing and Thawing of gMSC (Registered Trademark) 1)

Each gMSC (registered trademark) 1 in the thawed-by-method-A group to the thawed-by-method-C group was cryopreserved in accordance with the foregoing section 2-6, and then thawed by the following warming method corresponding to the group.
Thawed-by-method-A group: thawing was carried out using a 37° C. water bath for 2.5 minutes.
Thawed-by-method-B group: thawing was carried out by carrying out, by a Tube-in-Tube method, mixing by inversion at 22° C. (room temperature) at about 60 rpm for 3 minutes.
Thawed-by-method-C group: thawing was carried out using a heat block having a temperature set at 37° C. for 4.5 minutes.

Then, each gMSC (registered trademark) 1 was digested and decomposed in accordance with the foregoing section 2-5 until a single-cell suspension was obtained, and the number of cells was measured by the method stated in the section 2-5.

(Comparison of the Number of Cells after Thawing)

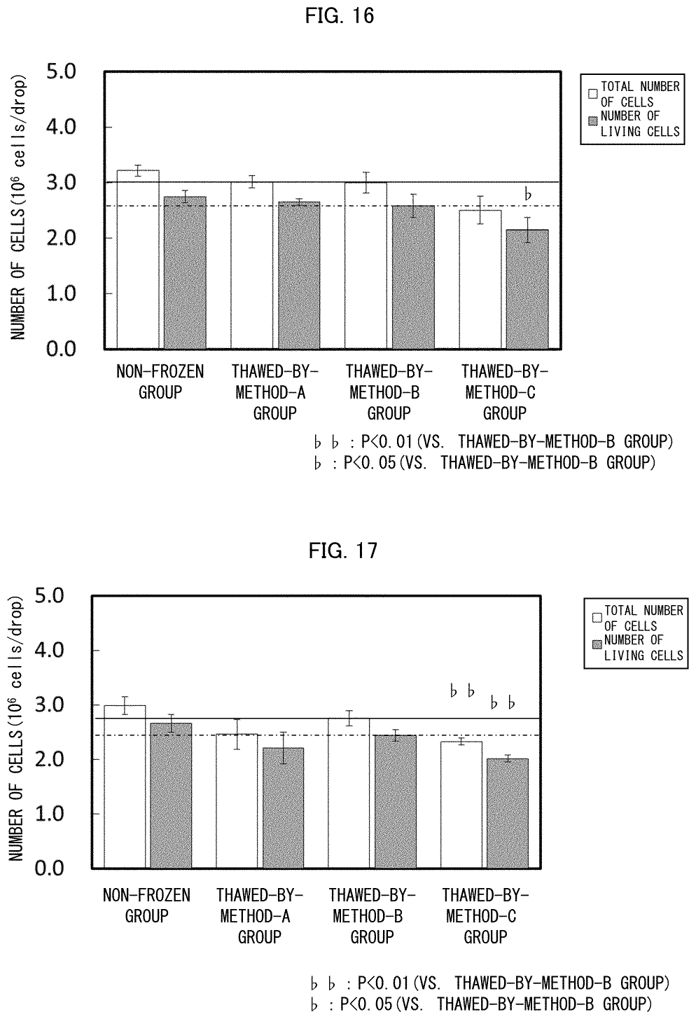

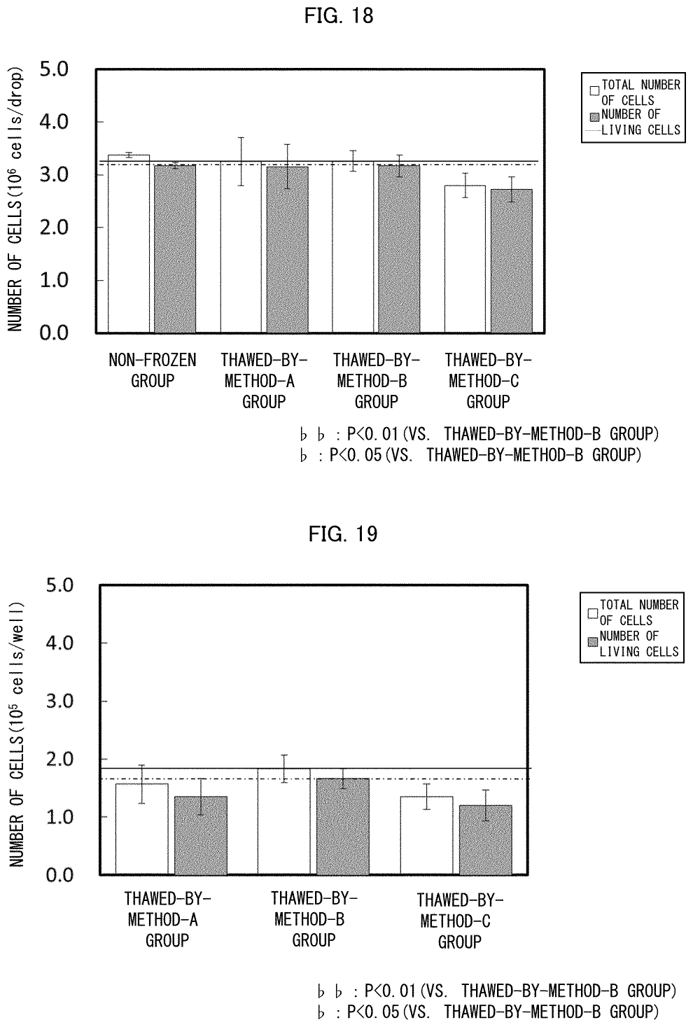

The result of thawing of strain 1 is shown in FIG. 16, the result of thawing of strain 2 is shown in FIG. 17, and the result of thawing of strain 3 is shown in FIG. 18. The category axis labels of the bar charts of FIGS. 16 to 18 correspond to “non-frozen group”, “thawed-by-method-A group”, “thawed-by-method-B group”, and “thawed-by-method-C group”, which are arranged in the order named from left, respectively. In each category, the white bar on the left represents “the total number of cells in a drop of gMSC (registered trademark) 1”, whereas the black bar on the right represents “the number of living cells in a drop of gMSC (registered trademark) 1”. The error bar for each bar represents standard deviation.

Note here that the total number of cells means the total number of cells (including living and dead cells) which were collected by the method stated in the foregoing section 2-5. The number of living cells means the number of living cells included in the cells which were collected by the method stated in the foregoing section 2-5. As used herein, the term “the cells which were collected” refers to cells which can be recognized as being cells in a usual cell measuring method such as a method using a cell counter, and does not include, e.g., residues resulting from breakage in the processes such as freezing, thawing, and collection.

There are two horizontal lines in the plot area in each of the charts in FIGS. 16 to 18. Of these horizontal lines, the solid line represents the total number of cells (average) in the thawed-by-method-B group in that chart, and the dot-dash line represents the number of living cells (average) in the thawed-by-method-B group in that chart. A comparison between the locations of these horizontal lines and the locations of the top ends of the bars corresponding to the other groups makes it possible to more easily know, at a glance, the difference between the total number of cells in each group and that of the thawed-by-method-B group and the difference between the number of living cells in each group and that of the thawed-by-method-B group.

Furthermore, there are the “♭♭” and “♭” symbols above some bars in the bar charts of FIGS. 16 to 18. The “♭♭” symbol means P<0.01 (vs. thawed-by-method-B group), and the “♭” symbol means P<0.05 (vs. thawed-by-method-B group). The test method here means independent Two-sample t test, and “P” means P value. For example, in a case where there is the “♭” symbol above the bar corresponding to the total number of cells in the thawed-by-method-C group, this means that “the total number of cells in the thawed-by-method-C group is greater than the total number of cells in the thawed-by-method-B group (P<0.05). In a case where there is the “♭” symbol above the bar corresponding to the number of living cells in the thawed-by-method-C group, this means that “the number of living cells in the thawed-by-method-C group is greater than the number of living cells in the thawed-by-method-B group (P<0.05)”. That is, the total number of cells is compared to the total number of cells, and the number of living cells is compared to the number of living cells.

The results shown in FIGS. 16 to 18 showed that both the total number of cells and the number of living cells after thawing are substantially the same between the 37° C. water bath method (thawed-by-method-A group) and the Tube-in-Tube method (thawed-by-method-B group). The results also showed that the total number of cells and the number of living cells after thawing in the case of the 37° C. water bath method (thawed-by-method-A group) and those in the case of the Tube-in-Tube method (thawed-by-method-B group) are both substantially the same as the total number of cells and the number of living cells in the non-frozen group.

The results also showed that the total number of cells and the number of living cells after thawing tend to be greater in the cases of Tube-in-Tube method (thawed-by-method-B group) and the 37° C. water bath method (thawed-by-method-A group) than in the case of the 37° C. heat block method (thawed-by-method-C group). These results showed that the cells in the case of the 37° C. heat block method (thawed-by-method-C group) tend to be damaged more than in the cases of the 37° C. water bath method (thawed-by-method-A group) and the Tube-in-Tube method (thawed-by-method-B group).

(Proliferating Ability (Start of Logarithmic Growth) of Thawed Cells)

It is generally known that cells immediately after freezing/thawing have a temporarily decreased proliferating ability. In view of this, cells were collected from the foregoing frozen-thawed gMSC (registered trademark) 1, and cells after one subculture were subjected to a comparison of the proliferating ability (start of logarithmic growth) of the cells immediately after thawing. Specifically, cells were obtained from each of the thawed-by-method-A to thawed-by-method-C groups (which had been subjected to the foregoing freezing/thawing and which had been digested and decomposed in accordance with the section 2-5 until a single-cell suspension was obtained), seeded again onto a 6 well plate, and were subjected to monolayer culture for a certain period of time. Then, the total number of cells and the number of living cells in the thawed-by-method-A group, those in the thawed-by-method-B group, and those in the thawed-by-method-C group were compared.

The number of cells seeded again into each well was 50,000. The cells seeded again into the wells are those collected from a single piece of gMSC (registered trademark) 1, that is, the cells are those which are derived from the same strain and which have been thawed by the same warming method. The conditions under which the monolayer culture was carried out are substantially the same as those of the foregoing section 2-2, and the culture time was fixed to 5 days for each group. The sample size in each group was as follows: thawed-by-method-A group (N=3); thawed-by-method-B group (N=3); and thawed-by-method-C group (N=3).

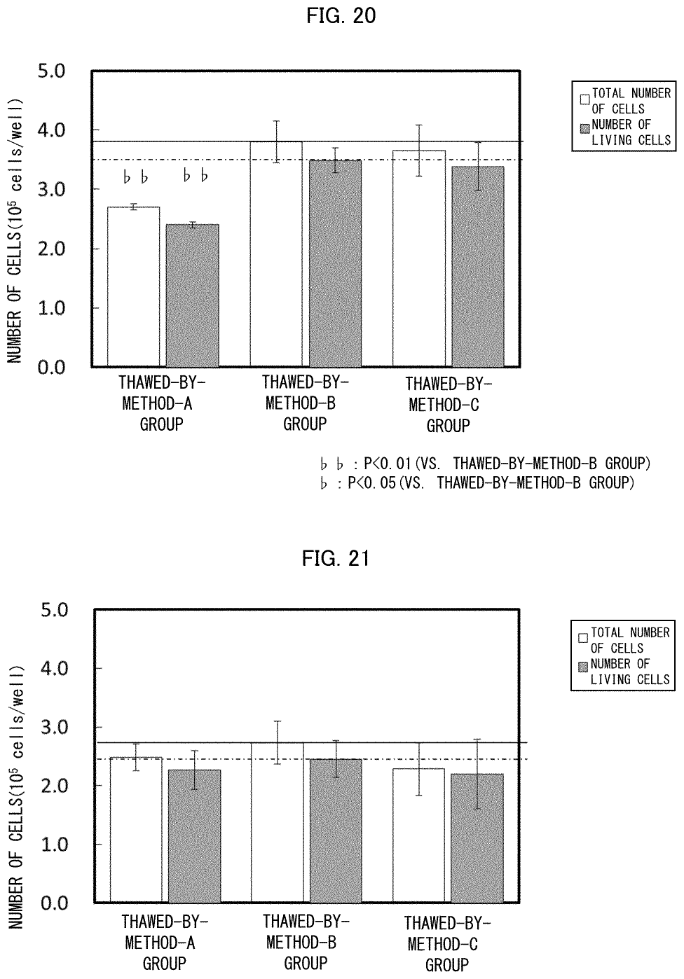

The proliferating ability of cells of strain 1 is shown in FIG. 19, the proliferating ability of cells of strain 2 is shown in FIG. 20, and the proliferating ability of cells of strain 3 is shown in FIG. 21. The legends in the bar charts of FIGS. 19 to 21 are as defined for FIGS. 16 to 18. These results showed that, in a case where the cells collected by the Tube-in-Tube method (thawed-by-method-B group) are seeded again, subjected to one subculture and collected, both the total number of cells and the number of living cells are greater than in the case of using cells collected from the thawed-by-method-A group or the thawed-by-method-C group. It was therefore found that the cells thawed using the Tube-in-Tube method (thawed-by-method-B group) are equivalent to or better than the cells thawed using the other two methods, in terms of the proliferating ability immediately after re-seeding of the thawed cells, i.e., start of logarithmic growth immediately after thawing.

[3-2: Comparison Between Non-Contact Tube-in-Tube Method and Conventional Method]

For evaluation of the effects of the non-contact Tube-in-Tube method, pieces of gMSC (registered trademark) 1 prepared and cryopreserved by the same method were warmed by different warming methods for comparison of cell performance. In this example, thawing was carried out by the following warming methods: non-contact Tube-in-Tube method (see “thawing method D” below); and 37° C. water bath method (see “thawing method A” below).

Note that, since gMSC (registered trademark) 1 is a three-dimensional cell mass, for measuring the number of cells, it is necessary to digest and decompose the cell mass until a single-cell suspension is obtained, as stated in the foregoing section 2-5. Therefore, a group whose number of cells was counted in accordance with the foregoing section 2-5 without carrying out a freezing operation (see “non-frozen group” below) was prepared as a control group which means “cells before freezing”. The details of the experiment are as follows.

(Preparation and Grouping of gMSC (Registered Trademark) 1)

A strain of MSCs established based on the foregoing section 2-1 was processed into gMSC (registered trademark) 1 in accordance with the methods stated in the foregoing sections 2-2 to 2-4, similarly to the foregoing section 3-1. Such gMSC (registered trademark) 1 was grouped into three groups consisting of non-frozen group, thawed-by-method-A group, and thawed-by-method-D group. The sample size in each group was as follows: non-frozen group (N=3), thawed-by-method-A group (N=3), and thawed-by-method-D group (N=3).

Each gMSC (registered trademark) 1 in the non-frozen group was digested and decomposed in accordance with the foregoing section 2-5 until a single-cell suspension was obtained, and the number of cells was measured under the condition of the section 2-5.

(Freezing and Thawing of gMSC (Registered Trademark) 1)

Each gMSC (registered trademark) 1 in the thawed-by-method-A group or the thawed-by-method-D group was cryopreserved in accordance with the foregoing section 2-6, and then thawed by the following warming method corresponding to the group.
Thawed-by-method-A group: thawing was carried out using a 37° C. water bath for 2.5 minutes.
Thawed-by-method-D group: thawing was carried out, by a non-contact Tube-in-Tube method, by carrying out mixing by inversion at 22° C. (room temperature) at about 60 rpm for 3 minutes.

Then, each gMSC (registered trademark) 1 was digested and decomposed in accordance with the foregoing section 2-5 until a single-cell suspension was obtained, and the number of cells was measured by the method stated in the section 2-5.

(Comparison of the Number of Cells after Thawing)

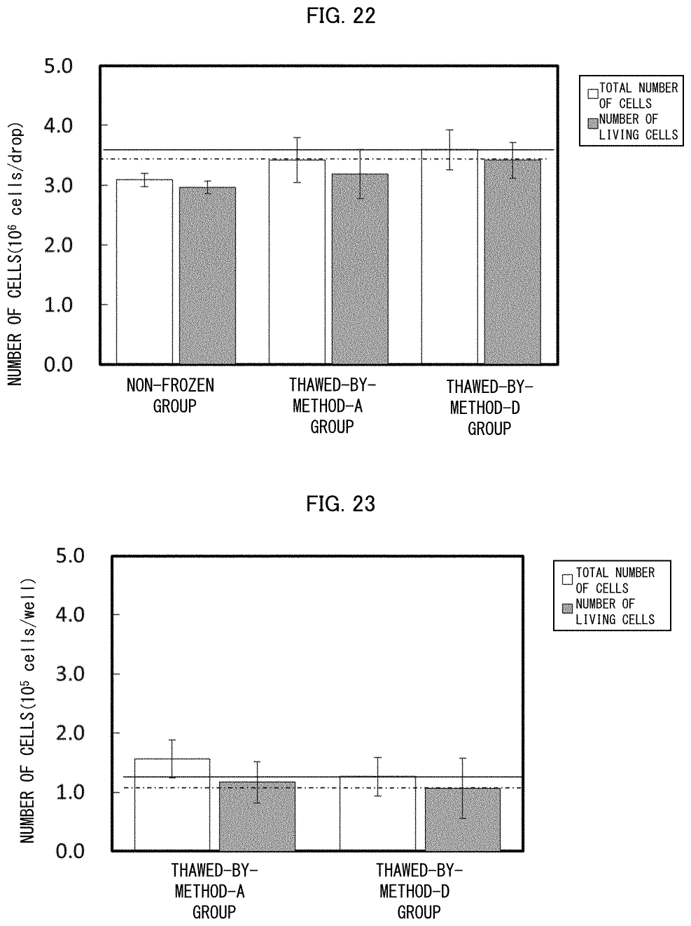

FIG. 22 shows the total number of cells and the number of living cells after thawing. The legends in FIG. 22 are substantially the same as those in FIGS. 16 to 21, except that, of the two horizontal lines in the plot area, the solid line represents the total number of cells (average) in the thawed-by-method-D group, whereas the dot-dash line represents the number of living cells (average) in the thawed-by-method-D group. The results in FIG. 22 showed that the cells thawed using the non-contact Tube-in-Tube method (thawed-by-method-D group) are equivalent to or better than the 37° C. water bath method (thawed-by-method-A group), in terms of both the total number of cells and the number of living cells.

(Proliferating Ability (Start of Logarithmic Growth) of Thawed Cells)

It is generally known that cells immediately after freezing/thawing have a temporarily decreased proliferating ability. In view of this, cells were collected from the foregoing frozen-thawed gMSC (registered trademark) 1, and cells after one subculture were subjected to a comparison of the proliferating ability (start of logarithmic growth). Specifically, cells were obtained from each of the thawed-by-method-A and thawed-by-method-D groups (which had been subjected to the foregoing freezing/thawing and which had been digested and decomposed in accordance with the section 2-5 until a single-cell suspension was obtained), seeded again onto a 6 well plate, and were subjected to monolayer culture for a certain period of time. Then, the total number of cells and the number of living cells in the thawed-by-method-A group and those in the thawed-by-method-D group were compared.

The number of cells seeded again into each well was 50,000. The cells seeded again into the wells are those collected from a single piece of gMSC (registered trademark) 1, that is, the cells are those which are derived from the same strain and which have been thawed by the same warming method. The conditions under which the monolayer culture was carried out are substantially the same as those of the foregoing section 2-2, and the culture time was fixed to 5 days for each group. The sample size in each group was as follows: thawed-by-method-A group (N=3); and thawed-by-method-D group (N=3).

FIG. 23 shows the proliferating ability of thawed cells. The legends in the bar chart of FIG. 23 are as defined for FIG. 22. The data showed that the cells thawed using the non-contact Tube-in-Tube method (thawed-by-method-D group) are equivalent to the cells thawed using the 37° C. water bath method (thawed-by-method-A group) in terms of the start of logarithmic growth.

[3-3: Comparison Between Non-Contact Tube-in-Tube Method and Conventional Method (2)]

This section discusses the results obtained by further evaluating the effectiveness of the thawing method D with use of a homogeneous cell mass (human skin fibroblast and human adipose derived MSC) instead of gMSC (registered trademark) 1.

(Establishment and Culture of Human Adipose Derived MSCs)

Adipose tissue provided from a related medical institution, through an appropriate ethical review and with patient's consent, was measured for its wet weight. The adipose tissue was transferred into a centrifuge tube having placed therein 10 mL of 10 μg/mL gentamicin (Nichi-Iko Pharmaceutical)-containing DMEM (SIGMA, D6046), and washed. The adipose tissue was transferred to another centrifuge tube having placed therein 10 mL of gentamicin-containing DMEM, washed again, and then taken from the centrifuge tube onto a container. The washed adipose tissue was cut into tissue fragments of not greater than 5 mm with use of sterilized scissors, digested with a 0.4% collagenase solution (Worthington Biochemical Corporation) at 37° C. for 1.5 hours, then subjected to filtration using a 100 μm mesh (Greiner Bio-One International GmbH), and collected in another 50 mL centrifuge tube. Then, centrifugation was carried out, a supernatant was removed, and the cells were suspended in STK (registered trademark) 1 (serum-free culture medium for primary MSC establishment, DS Pharma Biomedical Co., Ltd.). A part of the cell suspension was stained with 0.4% trypan blue (Thermo Fisher Scientific Inc.), and the number of living cells and the number of dead cells were counted. The cell suspension was diluted with STK (registered trademark) 1 until a seeding density of 5000 cells/cm² (surface area of culture plate) was reached, seeded onto a 150 cm² dish (SUMITOMO BAKELITE CO., LTD.), and culture was carried out under a condition in which CO₂ concentration was 5% and temperature was 37° C. for 14 days (culture medium was changed on day 5, day 8, and day 11). The same operations as described in the sections 2-2 and 2-3 were used to culture human adipose derived MSCs (A31, P4, hereinafter referred to as “ADMSCs”).

(Preparation, Freezing, and Thawing of Homogeneous Cell Mass)

The ADMSCs were cultured in an STK (registered trademark) 2 culture medium, and human skin fibroblasts (NHDF, P14, hereinafter referred to as “NHDFs”) obtained from Lonza Japan Ltd. were cultured in a DMEM culture medium containing 10% FBS, each under a condition in which CO₂ was 5% and temperature was 37° C. After collection (after detachment and washing), the cell suspension was transferred to a 2 mL cryogenic vial having placed therein 1 mL of a cell preservation solution. The cell preservation solution for the ADMSCs is CELLBANKER2 (Nippon Zenyaku Kogyo Co., Ltd.), and the cell preservation solution for the NHDF is CellBanker1 (Nippon Zenyaku Kogyo Co., Ltd.). Each cell suspension was frozen at −80° C. and, after one week of freezing, cell masses thawed by the thawing method A (thawed-by-method-A group (N=3)) and cell masses thawed by the thawing method D (thawed-by-method-D group (N=3)) were obtained. The details of the “thawing method A” and the “thawing method D” are provided in the section 3-1.

Equal aliquots of cell suspension were taken from samples of the thawed-by-method-A group and the thawed-by-method-D group, the taken cell suspensions were stained with trypan blue, and the total number of cells and the number of living cells contained in each of the stained cell suspensions were counted.

(Comparison of the Number of Cells after Thawing)

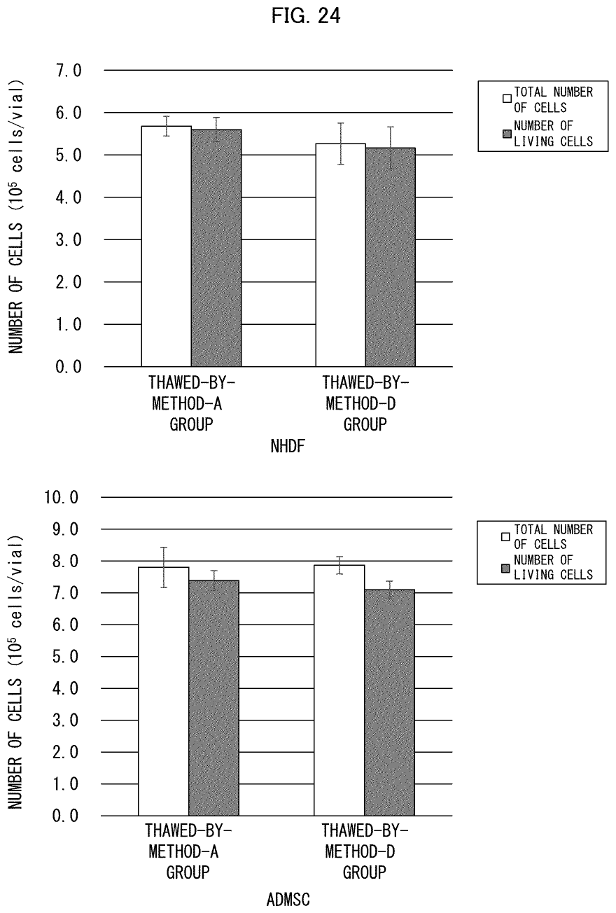

FIG. 24 shows the total number of cells and the number of living cells after thawing for NHDFs (upper panel) and those for ADMSCs (lower panel). As shown in FIG. 24, the thawed-by-method-D group was not significantly inferior to the thawed-by-method-A group in terms of a reduction in the total number of cells and a reduction in the number of living cells.

(Comparison of Proliferating Ability (Start of Logarithmic Growth) of Thawed Cells)

Cells contained in each of the cell suspensions, from which the foregoing aliquots had been taken, were evaluated for their proliferating ability. DMEM was added to each cell suspension, cells were washed, and then the cells were subjected to centrifugation. The NHDFs after subjected to centrifugation (NHDFs of both the thawed-by-method-A group and the thawed-by-method-D group) were suspended again in a DMEM culture medium containing 10% FBS. The ADMSCs after subjected to centrifugation (ADMSCs of both the thawed-by-method-A group and the thawed-by-method-D group) were suspended again in an STK (registered trademark) 2 culture medium. The suspended NHDFs and ADMSCs of the thawed-by-method-A group and the thawed-by-method-D group were seeded onto 6 well plates at 5×10⁴ cells/well. The 6 well plate having NHDFs seeded thereon was allowed to stand in the culture conditions of 5% CO₂ and 37° C. for 5 days. The 6 well plate having ADMSCs seeded thereon was allowed to stand in the culture conditions of 5% CO₂ and 37° C. for 7 days.

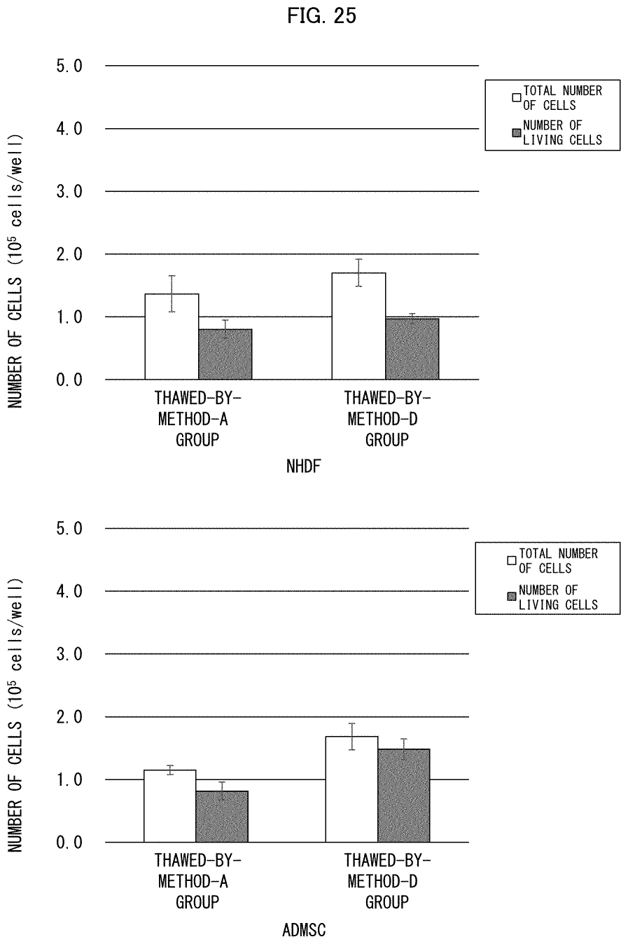

FIG. 25 shows the total number of cells and the number of living cells after thawing and culture for NHDFs (upper panel) and those for ADMSCs (lower panel). As shown in FIG. 25, the thawed-by-method-D group was not inferior in the proliferating ability of the cells, and was slightly better than the thawed-by-method-A group in terms of the total number of cells and the number of living cells (average).

As has been described, it was found that the thawing method D in accordance with an example of the present invention makes it possible to thaw a cryopreserved homogeneous cell mass (for example, established cell strain) with effectiveness (the number of living cells and proliferating ability) equivalent to or better than that in the case of the thawing method A (conventional method).

### INDUSTRIAL APPLICABILITY

The present invention is capable of providing a safe, valuable graft treatment material, and therefore can be suitably used for regenerative medicine such as a graft treatment.

### REFERENCE SIGNS LIST

1, 10 warming container
2 heat medium container
3 opening (inlet)
4 heat medium
5 biological sample container
11 resin film (positioning part)

1. A method of warming a biological sample, comprising the steps of:
i) putting a biological sample container into a heat medium container, the biological sample container having a biological sample disposed therein, the heat medium container having a heat medium disposed therein;
ii) closing an inlet of the heat medium container to prevent the heat medium from leaking out of the heat medium container, the inlet being an inlet through which the heat medium is injected into the heat medium container; and
iii) moving the heat medium in the heat medium container with the inlet closed.

2. The method as set forth in claim 1, wherein: step ii) is carried out after step i); and step iii) is carried out by moving the heat medium container.

3. The method as set forth in claim 1, wherein step iii) is carried out by holding and shaking the heat medium container by hand.

4. The method as set forth in claim 1, wherein step iii) is carried out under a condition in which the heat medium has a temperature of not lower than 20° C. and not higher than 40° C.

5. The method as set forth in claim 4, wherein step iii) is carried out under a condition in which the heat medium has a temperature of not lower than 20° C. and not higher than 27° C.

6. The method as set forth in claim 1, wherein the biological sample is at least one selected from the group consisting of cells, cell masses, tissue, and tissue fragments.

7. The method as set forth in claim 1, wherein the heat medium is at least one selected from the group consisting of water, isotonic solutions, and water which has an antibacterial agent dissolved therein.

8. A warming container for warming a biological sample, comprising:
a heat medium container configured to have a heat medium disposed therein and have a biological sample container disposed therein, the biological sample container being configured to have a biological sample disposed therein; and
a positioning part which is attached to the heat medium container and which is configured to keep the biological sample container in position.

9. The warming container as set forth in claim 8, wherein the positioning part is provided inside the heat medium container.

10. The warming container as set forth in claim 8, wherein:
the heat medium container is in the form of a pouch;
the positioning part constitutes an outside surface of the heat medium container; and
the warming container is configured such that the heat medium container in the form of a pouch is folded around the biological sample container to have the biological sample container disposed in a space defined by the folded heat medium container and to keep the biological sample container in position.

11. The warming container as set forth in claim 8, wherein the positioning part is a resin film.

12. The warming container as set forth in claim 8, wherein:
the heat medium container is a tubular structure which has opposite ends one of which is a closed end and the other of which is an open end; and
the warming container further includes a lid configured to close the open end.

13. The warming container as set forth in claim 12, wherein the tubular structure has, in a cross section perpendicular to a longitudinal direction of the tubular structure, a diameter of not less than 5 mm and not more than 200 mm.

14. The warming container as set forth in claim 8 wherein the warming container has no heating elements therein.

15. A kit for warming a biological sample, comprising a heat medium container configured to have a heat medium and a biological sample container disposed therein, wherein:
the heat medium container includes a positioning part configured to keep the biological sample container in position; and
the biological sample container is configured to have a biological sample disposed therein.

16. The kit as set forth in claim 15, wherein:
the biological sample container has disposed therein a biological sample preserved at low temperature; and
the kit further comprises a cold keeper configured to keep the biological sample in a cold state.

17. The kit as set forth in claim 15, further comprising a wash liquid for use in washing the biological sample.

18. The kit as set forth in claim 15, wherein:
the biological sample container has a biological sample disposed therein; and
the biological sample container has been sterilized and is in a hermetically sealed container.
